(19) United States
(12) Patent Application Publication
(10) Pub. No.: US 2016/0038526 A1
(43) Pub. Date: Feb. 11, 2016

(54) TREATMENT OF PROSTATE CANCER

(71) Applicants: THOMAS JEFFERSON UNIVERSITY, Philadelphia, PA (US); GEORGETOWN UNIVERSITY, Washington, DC (US)

(72) Inventors: Marja Tuuli Nevalainen, Gladwyne, PA (US); Nagarajan Pattabiraman, North Potomac, MD (US)

(21) Appl. No.: 14/421,584

(22) PCT Filed: May 10, 2013
(86) PCT No.: PCT/US13/40472

§ 371 Date: Feb. 13, 2015

Related U.S. Application Data
(60) Provisional application No. 61/683,917, filed on Aug. 16, 2012.

(51) Int. Cl. A61K 31/7076; A61N 5/10; A61K 45/06

(52) U.S. Cl. CPC A61K 31/7076; A61K 45/06; A61N 5/10

(57) ABSTRACT

Prostate cancer is treated by administration of N⁶-benzyladenosine-5′-monophosphate or pharmaceutically acceptable salt thereof.

### CROSS-REFERENCE TO RELATED APPLICATION

The benefit of the filing date of U.S. Provisional Patent Application No. 61/683,917, filed Aug. 16, 2012, is hereby claimed. The entire disclosure of the aforesaid application is incorporated herein by reference.

### FIELD OF THE INVENTION

The invention relates to the treatment of prostate cancer.

### BACKGROUND OF THE INVENTION

### Prostate Cancer

Prostate cancer is the third leading cause of cancer deaths among men in the United States. The number of new cases of prostate cancer, estimated at more than 220,000 per year in 2005, is expected to increase to more than 380,000 by 2025 because of the aging male population (Scardino, N Engl J Med 2003, 349:297-299). Organ-confined primary prostate cancer is treated by surgery, radiation, hormone therapy, or combinations of these treatment modalities, depending on the age, operability of the patient and tolerance for the specific treatment-related side-effects. Duration of response to hormonal therapy is limited and prostate cancers inevitably become castration-resistant and metastatic, a stage of the disease for which there is no curative treatment. For a significant fraction of prostate cancers, the existing therapies only provide a temporary relief of the symptoms, while the castration-resistant and/or metastatic forms of prostate cancer develop. Currently, there are no effective pharmacological therapies for advanced prostate cancer (Pestell R G, Nevalainen, M. T.: Prostate Cancer: Signaling Networks, Genetics and New Treatment Strategies. Totowa, Human Press, 2008).

Stat5 is one of 7 members of the Stat (Signal Transducer and Activation of Transcription) family of transcription factors in mammals, and consists of two distinct, but highly homologous, proteins, the 94-kDa Stat5a and 92-kDa Stat5b factors (Ihle et al., Curr Opin Cell Biol 2001, 13:211-217). The isoforms are encoded by separate genes (Id.) Stat5a and Stat5b (hereafter referred to collectively as “Stat5a/b”) are latent cytoplasmic proteins that act as both cytoplasmic signaling proteins and nuclear transcription factors. Stat5a and Stat5b become activated by phosphorylation on residue Tyr694 and Tyr 699, respectively, in the C-terminal domain predominantly by Janus tyrosine kinase-2 (Jak2), which is preassociated with the cytoplasmic domain of the prolactin (Prl) receptor (PrlR). After phosphorylation, Stat5a/b homo- or hetero-dimerize and translocate to the nucleus where they bind to specific Stat5a/b response elements of target gene promoters (Id.)

Stat5 proteins are composed of five structurally and functionally conserved domains. The N-terminal domain stabilizes interactions between two Stat5 dimers to form tetramers for maximal transcriptional activation of weak promoters (John et al., Mol Cell Biol 1999, 19:1910-1918). The coiled-coil domain facilitates protein-protein interactions (Chen et al., Cell 1998, 93:827-839; Becker et al., Nature 1998, 394:145-151). The DNA-binding domain recognizes members of the GAS family of enhancers (Levy et al., Nat Rev Mol Cell Biol 2002, 3:651-662). The SH2 domain contains a binding pocket for the phosphotyrosine residue of another Stat5 molecule thereby mediating both receptor-specific recruitment followed by phosphorylation and STAT dimerization (Kisseleva et al., Gene 2002, 285:1-2425). The carboxy terminus carries a transactivation domain (TAD), which binds critical co-activators and is directly involved in initiation of transcription (Levy et al., supra; Darnell, Science 1997, 277:1630-1635).

Stat5a/b is critical for the viability of Stat5a/b-positive human prostate cancer cell lines in culture and for growth of human prostate cancer xenograft tumors in nude mice (Ahonen et al., J Biol Chem 2003, 278:27287-27292; Dagvadorj et al., Endocrinology 2007, 148:3089-3101; Dagvadorj et al., Clin Cancer Res 2008, 14:1317-1324). Adenoviral expression of a dominant-negative (DN) mutant of Stat5a, blocking both Stat5a and Stat5b, induced massive and rapid apoptotic death of human prostate cancer cells in culture. Id. Inhibition of Stat5a/b by antisense oligo-nucleotides or RNA interference also induced rapid apoptotic death of prostate cancer cells (Dagvadorj et al. Clin Cancer Res, supra), blocked human prostate cancer xenograft tumor growth (both subcutaneous and orthotopic) in nude mice, and down-regulated BclXL and Cyclin-D1 protein levels in prostate cancer cells (Id.).

Nuclear Stat5 in prostate cancer correlates with loss of differentiation. The levels of active Stat5a/b are increased in human prostate cancer but not in normal human prostate epithelium (Ahonen et al., supra). The levels of active Stat5a/b are elevated in prostate cancers of high histological grades (n=114, P<0.0001) (Li et al., Cancer Res 2004, 64:4774-4782), a finding later confirmed in a second independent study using material of 357 human prostate cancers (P=0.03) (Li et al., Clin Cancer Res 2005 11:5863-8). Active Stat5a/b levels are also elevated in the majority of castration-resistant recurrent human prostate cancers (Tan et al., Cancer Res 2008, 68:236-248). Increased active Stat5a/b in primary prostate cancer predicted early disease recurrence after initial treatment by radical prostatectomy (Li et al., Clin Cancer Res 2005, supra). When only prostate cancers of intermediate Gleason grades were analyzed, increased active Stat5a/b remained an independent predictive marker of early recurrence of prostate cancer (Id.).

Stat5a/b activation is strongly associated with high histological grade of prostate cancer (Li et al., Cancer Res 2004, 64:4774-4782; Li et al., Clin Cancer Res 2005, 11:5863-5868), but Stat5a/b is not active in normal prostate epithelium (Ahonen et al., supra). Stat5a/b activation in primary prostate cancer predicts early disease recurrence (Li et al., Clin Cancer Res 2005, 11:5863-5868). Nuclear Stat5a/b is over-expressed in castration-resistant clinical prostate cancers (Tan et al., Cancer Res 2008, 68:236-248; Tan et al., Endocr Relat Cancer 2008, 15:367-390). Stat5a/b is active in 95% of clinical hormone-refractory human prostate cancers, and synergizes with androgen receptor (AR) in prostate cancer cells (Tan et al., Cancer Res 2008, supra).

Stat5 is involved in induction of metastatic behavior of human prostate cancer cells. Nuclear Stat5 levels are increased in 61% of distant metastases of clinical prostate cancer (Gu et al., Endocrine-Related Cancer 2010, 17(2):481-493). Stat5 increased metastases formation of prostate cancer cells to the lungs of nude mice by 11-fold in an experimental in vivo metastases assay (Id.). Active Stat5 induced migration and invasion of prostate cancer cells, which was accompanied by Stat5-induced re-arrangement of the microtubule network. Active Stat5 expression was associated with decreased cell-surface E-cadherin levels, while heterotypic adhesion of prostate cancer cells to endothelial cells was stimulated by active Stat5 (Id.)

US Pat. Pub. 2007/0010468A1 describes methods and compositions for the inhibition of Stat5 in prostate cancer, and describes the treatment of prostate cancer by inhibition of Stat5. Transfection of the androgen-independent human prostate cell line CWR22Rv with an adenovirus vector carrying a dominant-negative Stat5a gene (AdNStat5) is described. A dose-dependent effect of the expression of said DNStat5 on prostate cell viability was observed. Microscopic assessment of the effect of AdDNStat5 on CWR22Rv cell viability confirmed extensive cell death following expression of DNStat5. AdDNStat5 also induced cell death in the androgen-sensitive human prostate cancer cell line, LnCap. Apoptotic cell death of prostate cancer cells expressing DNStat5 was verified by DNA fragmentation analysis and cell cycle analysis. Importantly, Stat inhibition kills both AR-positive and AR-negative prostate cancer cells indicating that both AR-independent and AR-associated pathways mediate the effects of Stat on prostate cancer cell viability (Gu et al., Am. J. Pathology 2010, 176(4):1959-1972).

Thus, blocking Stat5 activity was observed to induce apoptosis of prostate cancer cells.

While US Pat. Pub. 2007/0010468A1 and other sources discussed above suggest that interfering with the biological activity of Stat5 in human prostate is a therapeutic approach, what is needed is a small molecule that would be effective in inhibiting Stat5 activation and its biological activities, to inhibit growth of prostate tumor cells.

### SUMMARY OF THE INVENTION

Provided is a method of treating prostate cancer in a male in need of such treatment comprising administering to the male a therapeutically effective amount of N⁶-benzyladenosine-5′-monophosphate, or pharmaceutically acceptable salt thereof.

In another embodiment, a method of inhibiting prostate cancer cell growth is provided, comprising contacting prostate cancer cells with an amount of N⁶-benzyladenosine-5′-monophosphate, or pharmaceutically acceptable salt thereof, effective to inhibit such cell growth.

In another embodiment, a method of inhibiting prostate cancer cell growth in a male comprises administering to the male in need of treatment a therapeutically effective amount of N⁶-benzyladenosine-5′-monophosphate, or pharmaceutically acceptable salt thereof. The growth of prostate cancer cells in the male is inhibited by such administration.

The prostate cancer treated may be, for example, organ-confined primary prostate cancer, locally invasive advanced prostate cancer, metastatic prostate cancer, castration-resistant prostate cancer or recurrent castration-resistant prostate cancer. Metastatic prostate cancer is characterized by prostate cancer cells that are no longer organ-confined. Recurrent castration-resistant prostate cancer is prostate cancer that does not respond to androgen-deprivation therapy or prostate cancer that recurs after androgen-deprivation therapy.

In another embodiment, a method of treating prostate cancer in a male in need of such treatment is provided comprising administering to the male a therapeutically effective amount of N⁶-benzyladenosine-5′-monophosphate, or pharmaceutically acceptable salt thereof, and at least one of radiation therapy and chemotherapy with an other chemotherapeutic agent effective against prostate cancer. By “other chemotherapeutic agent effective against prostate cancer” is meant a chemotherapeutic agent, other than N⁶-benzyladenosine-5′-monophosphate, or pharmaceutically acceptable salt thereof, that is effective in treating prostate cancer. N⁶-benzyladenosine-5′-monophosphate, and pharmaceutically acceptable salts thereof, are referred to in this context as “primary anti-prostate cancer agent”. In some embodiments, the other chemotherapeutic agent is selected from the group consisting of docetaxel, mitoxantrone, estramustine, doxorubicin, etoposide, vinblastine, paclitaxel, carboplatin, and vinorelbine, and combinations thereof. In some embodiments, the other chemotherapeutic agent is administered simultaneously with the said primary anti-prostate cancer agent. In other embodiments, the other chemotherapeutic agent is administered serially with said primary anti-prostate cancer agent. In one embodiment of simultaneous administration, the primary anti-prostate cancer agent and the other chemotherapeutic agent are contained in the same dosage form.

In some embodiments, the male treated according to the above methods is a male human being.

In another embodiment, a method for treatment of prostate cancer comprises administering to a male in need of such treatment a therapeutically effective amount of N⁶-benzyladenosine-5′-monophosphate, or pharmaceutically acceptable salt thereof, and an androgen ablation therapy. In one embodiment, the androgen ablation therapy comprises androgen deprivation therapy. In another embodiment, the androgen ablation therapy comprises administration of at least one luteinizing hormone releasing hormone agonist, at least one anti-androgen, or at least one inhibitor of androgenic steroid synthesis in the prostate. In another embodiment, the androgen ablation therapy may comprise a combination of drugs from two or all three of the aforementioned categories. For examples, the ablation therapy may comprise a combination of at least one luteinizing hormone releasing hormone agonist, and at least one anti-androgen, and/or at least one inhibitor of androgenic steroid synthesis.

In another embodiment, N⁶-benzyladenosine-5′-monophosphate, or pharmaceutically acceptable salt thereof, is used for treating prostate cancer.

### Abbreviations

The following abbreviation may be utilized in the text and the figures:

N6BAP or N⁶BAP: N⁶-benzyladenosine-5′-monophosphate.

### BRIEF DESCRIPTION OF THE FIGURES

FIG. 1A shows the domain structure of Stat5.

FIG. 1B shows a binding model of N⁶-benzyladenosine-5′-monophosphate (N6BAP), to the Stat5a SH2-domain.

FIG. 1C shows a binding model of to the Stat5a SH2-domain by stick model showing the SH2 dimer interface site of Stat5a. Sub-pockets P1, P2, P3 and P1′ of the SH2 interface are labeled.

FIG. 2A shows the effect of varying concentration of N6BAP on the transcriptional activity of Stat5a and Stat5b on the β-casein gene promoter in a luciferase reporter gene assay in the human prostate cancer cell line PC-3. The compound inhibited Stat5a and Stat5b transcriptional activity in a concentration-dependent manner.

FIG. 2B is similar to FIG. 2A, and shows the effect of varying concentrations of N6BAP on the transcriptional activity of Stat5a compared to the effect of five control compounds (“C3” through “C7”, respectively). The control compounds are similar in molecular weight to N6BAP, but unrelated in chemical structure. As shown in FIG. 2B, compounds C3 through C7 did not inhibit transcriptional activity of Stat5a on the 3-casein gene promoter in PC-3 cells.

FIG. 3A shows the result of a study demonstrating that N6BAP blocks dimerization of Stat5a/b in human prostate cancer cells. DMSO and the control compound C5 were included in the study as controls.

FIG. 3B shows that N6BAP inhibits phosphorylation of Stat5 after ligand (prolactin)-induced activation, due to N6BAP blocking the SH2-domain mediated docking of Stat5 to the prolactin-receptor Jak2-complex and subsequent phosphorylation of Stat5a/b. FIG. 3B also shows that N6BAP does not affect phosphorylation of Jak2 indicating that N6BAP is not an inhibitor of Jak2.

FIG. 4 shows the result of a study demonstrating that N6BAP blocks nuclear translocation of Stat5a in human prostate cancer cells after ligand (prolactin)-induced activation.

FIG. 5 shows the results of an electromobility shift study comparing the effect of N6BAP and the control compound C5 on the binding of Stat5a and Stat5b to DNA following ligand (prolactin)-induced activation.

FIG. 6 shows that N6BAP inhibits protein expression of Stat5-driven genes (cyclinD1, BclXL) in human prostate cancer cells.

FIG. 7 shows that N6BAP does not inhibit transcriptional activity of I1-6-induced Stat3. This demonstrates that N6BAP is specific for the SH2-domain of Stat5.

FIG. 8. shows that N6BAP does not block nuclear translocation of I1-6-induced Stat3, again indicating specificity of N6BAP for the SH2-domain of Stat5. As a positive control, it is shown that the Jak2-inhibitor AZD1480 blocks nuclear translocation of IL-6-induced Stat3 efficiently.

FIG. 9A shows the effect of N6BAP on the viability of human prostate CWR22Rv1 cells compared to untreated controls (Ctrl) at 8, 16, and 24 hours post-treatment.

FIG. 9B shows N6BAP-induced DNA fragmentation in CWR22Rv1 cells treated with 24 μM N6BAP, indicating that N6BAP-induced cell death is apoptotic

FIGS. 9C, 9D and 9E show that N6BAP decreases the number of viable cells of the following prostate cancer cell lines: 9C: CWR22Rv1 (72 h hours); 9D: LNCaP, (72 hours); and 9E: DU145 (72 hours).

FIGS. 10A-10C comprise a series of plots of another set of cell viability assays, showing that N6BAP did not affect the viability of the non-prostate solid tumor cell lines A549 (human lung cancer) and HT1080 (fibrosarcoma) (FIG. 10A); CAPAN (pancreatic) and HepG2 (hepatocellular) (FIG. 10B); and T47D (human breast cancer) and COS-7 (monkey kidney) (FIG. 10C).

FIG. 11A includes a plot of the volume of a tumor xenograft in nude mice from implantation of the human prostate cancer cell line CWR22Rv1. Mice were treated with the indicated doses of N6BAP, vehicle (hydroxycellulose, “HPC”), or received no treatment. N6BAP inhibited the growth of the xenograft tumor in a dose-dependent manner. Photographs of the treated animals are shown.

FIG. 11B shows tumor xenograft volumes for the individual tumors in all treatment groups which contributed to the data of FIG. 11A. Individual tumors are represented as columns.

FIG. 12A includes a plot of tumor cell viability in the tumor xenografts of FIG. 11, showing that N6BAP decreased the number of live epithelial cells, as determined by hematoxylin-eosin staining. The corresponding stained views are shown in the panels beneath the plot.

FIG. 12B includes a plot of apoptotic epithelial cells in the tumor xenografts of FIG. 11, as determined by in situ end labeling of fragmented by the TUNEL method. Cell viability indexes were determined by counting alive cells per total number of cells per view established in the no treatment group. TUNEL indexes were determined by counting epithelial cells with TUNEL-positive cells per total number of epithelial cells per view. The counts were averaged within the tumors in a given treatment group. Nucleotides incorporated into fragmented DNA were detected after incubation with anti-fluorescein antibody conjugated with peroxidase followed by visualization with 3,3-diaminobenzidine as a chromogen and methyl green as a counterstain. Stained views are shown in the panels beneath the plot in FIG. 12B. N6BAP increased apoptotic epithelial cells within the tumor xenografts.

FIG. 13A includes a plot of anti-Stat5a/b antibody staining of paraffin-embedded sections of the tumor xenografts of FIG. 11. The figure shows that N6BAP decreases the amount of nuclear Stat5 in prostate cancer xenograft tumors. Three microscopic views of each tumor (ten tumors per each treatment group) were photographed at 20× magnification. Nuclear Stat5 indexes were determined by counting epithelial cells with nuclear Stat5a/b immunostaining-positive cells per total number of epithelial cells per view. The counts were averaged within the tumors in a given treatment group. N6BAP decreased nuclear Stat5 expression in the prostate cancer xenograft tumors. Stained views are shown in the panels beneath the plot in FIG. 13A.

FIG. 13B is similar to FIG. 13A, but shows the results of anti-Stat3 antibody staining. The results show that N6BAP did not affect nuclear translocation of Stat3 in the tumor xenografts. Stained views are shown in the panels beneath the plot in FIG. 13B.

FIG. 14A includes a plot of N6BAP-induced death of prostate cancer epithelium in human clinical prostate cancers ex vivo in organ explant cultures. Localized prostate cancers from eight patients were cultured 7 days ex vivo in explant organ cultures in basal medium in the presence of N6BAP at indicated concentrations, or the control compound C5. The samples were positive for nuclear Stat5 prior to culture. Viability of the epithelial cells in the prostate cancer explants at the end of the cultures were scored by counting the viable epithelial cells per explant. The cultures responded to N6BAP by excessive loss of viable acinar epithelium. The stained views shown in the panels beneath the plot in FIG. 14A comprise a representative histology of one individual.

FIG. 14B shows the levels of nuclear active Stat5a/b determined by immunohistochemistry in human clinical prostate cancers at the end of organ explant cultures. Nuclear Stat5a/b indexes were determined by counting the Stat5-positive cells per 500 epithelial cells in the cultured explants in each treatment group. Intensive positive immunostaining for nuclear Stat5 in explants cultured in the presence of the control compound C5 is observed, while N6BAP reduced the levels of nuclear Stat5 expression. The stained views shown in the panels beneath the plot in FIG. 14A comprise a representative immunostaining of nuclear Stat5 in a human clinical prostate cancer that was responsive to N6BAP.

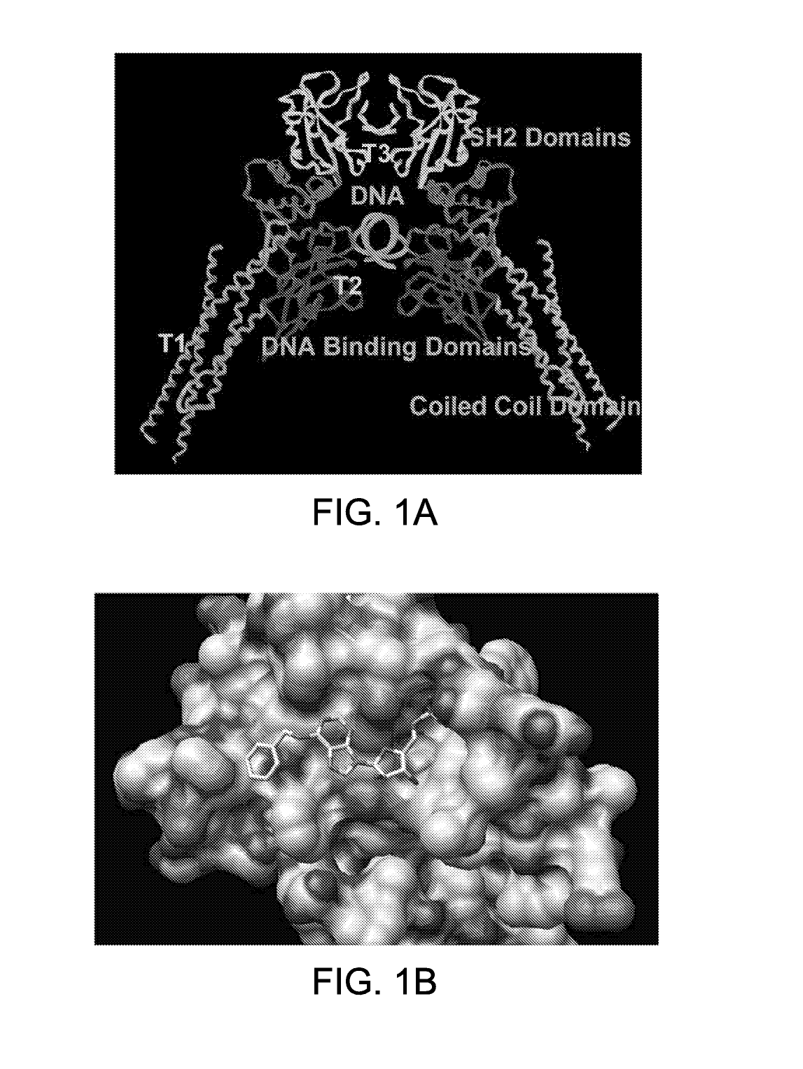

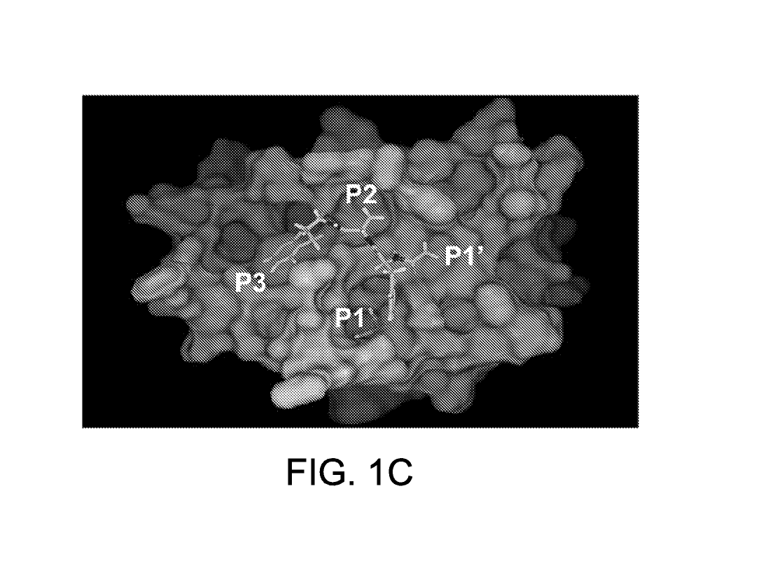

### DETAILED DESCRIPTION OF THE INVENTION

According to the present invention, N⁶-benzyladenosine-5′-monophosphate, or pharmaceutically acceptable salt thereof, inhibits the proliferation of prostate tumor cells, and causes their death, by inhibiting biological activities of Stat5a and/or Stat5b.

As used in the specification and the appended claims, the singular forms “a,” “an” and “the” include plural referents unless the context clearly dictates otherwise.

As used herein, the terms “treat” and “treatment” are used interchangeably and are meant to indicate a postponement of development of a disorder and/or a reduction in the severity of symptoms that will or are expected to develop. The terms further include ameliorating existing symptoms, preventing additional symptoms, and ameliorating or preventing the underlying metabolic causes of symptoms.

The expression “effective amount” or “therapeutically effective amount” when used to describe therapy to an individual suffering from prostate cancer, refers to the amount of a compound that inhibits the growth or proliferation of prostate cancer cells, or alternatively induces apoptosis of such cells, preferably resulting in a therapeutically useful and preferably selective cytotoxic effect on prostate cancer cells. In one embodiment, the prostate cancer cells are part of a prostate tumor.

As used herein, the term “subject” or “patient” refers to any animal (e.g., a mammal), including, but not limited to humans, non-human primates, rodents, and the like. Typically, the terms “subject” and “patient” are used interchangeably herein in reference to a human subject.

As envisioned in the present invention with respect to the disclosed compositions of matter and methods, in one aspect the embodiments of the invention comprise the components and/or steps disclosed herein. In another aspect, the embodiments of the invention consist essentially of the components and/or steps disclosed herein. In yet another aspect, the embodiments of the invention consist of the components and/or steps disclosed herein.

N⁶-benzyladenosine-5′-monophosphate has the following chemical structure:

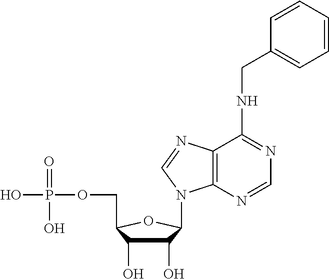

N⁶-benzyladenosine-5′-monophosphate may be prepared as described in the literature.

According to the present invention, it has been found that N⁶-benzyladenosine-5′-monophosphate blocks the transcriptional activity of Stat5a/b in prostate cancer cells, blocks the nuclear translocation of Stat5a/b in human prostate cancer cells, inhibits the dimerization of Stat5a/b in human prostate cancer cells, inhibits the binding of Stat5a/b to DNA in human prostate cancer cells, and/or induces death of human prostate cancer cell lines in vitro and in vivo.

In the activation cascade of Stat5a/b, the molecule first becomes phosphorylated at a conserved tyrosine residue in its C-terminus by an upstream tyrosine kinase (such as Jak2 or Src). Phosphorylation of Stat5a/b leads to dimerization of Stat5a/b, and the dimerized Stat5a/b translocates to the nucleus followed by binding of dimerized Stat5a/b to the promoters of its target genes to regulate transcription. Dimerization of Stat5a/b is required for Stat5a/b to bind DNA and exert is transcriptional activity. According to the present invention, N⁶-benzyladenosine-5′-monophosphate blocks Stat5a/b dimerization, as demonstrated in the assays described below.

Translocation of Stat5a/b to the nucleus is required in order for that molecule to exert its transcriptional activity. In the nucleus, Stat5a/b bind to specific Stat5a/b response elements of target gene promoters. According to the present invention, N⁶-benzyladenosine-5′-monophosphate blocks Stat5a/b translocation to the nucleus of prostate cancer cells after ligand (prolactin)-induced activation, and inhibits the binding of Stat5a/b to the Stat5 DNA consensus sequence in those cells. N⁶-benzyladenosine-5′-monophosphate inhibits proliferation of prostate cancer cells, and induces apoptosis of those cells, as demonstrated in the assays described below.

In some embodiments, Stat5a/b inhibition selectively targets prostate cancer cells but not normal prostate epithelial cells or cells of other organs.

N⁶-benzyladenosine-5′-monophosphate and pharmaceutically acceptable salts thereof are useful to provide therapy for primary, recurrent and metastatic prostate cancer. The compounds may also be used for adjuvant therapy for prostate cancer after surgery and for sensitization of prostate cancer to radiation and chemotherapy, e.g., docetaxel chemotherapy. In addition, the compounds may be used for prevention of metastatic progression of the initial organ-confined primary prostate cancer after diagnosis and the initial treatment. The compounds are also useful for treating recurrent castration-resistant prostate cancer and advanced disseminated prostate cancer.

Nuclear Stat5 levels are increased in 61% of distant metastases of clinical prostate cancer, and Stat5 promotes metastatic behavior of prostate cancer cells (Gu et al., Endocr Relat Cancer 2010; 17:481-493). Thus, N⁶-benzyladenosine-5′-monophosphate and pharmaceutically acceptable salts are useful in treating metastatic prostate caner, by inhibiting Stat5a/b activity.

During hormonal therapy, androgen-independent tumor cells eventually emerge, leading to clinical relapse, and the condition known as castration-resistant human prostate cancer. There are no effective treatments available for this condition. Stat5a/b is active in 95% of clinical castration-resistant human prostate cancers (Tan et al., Cancer Res 2008; 68(1):236-48). However, Stat5a/b has been shown to be active in 95% of clinical castration-resistant human prostate cancers (Tan et al., Cancer Res 2008; 68(1):236-48), thus presenting a target for therapy. Accordingly, another aspect of the present invention is the treatment of castration-resistant prostate cancer, by administration of N⁶-benzyladenosine-5′-monophosphate or pharmaceutically acceptable salt to a male in need of such treatment.

N⁶-benzyladenosine-5′-monophosphate may be converted to a salt for use according to the present invention. The term “pharmaceutically acceptable salt” refers to salts which possess toxicity profiles within a range that affords utility in pharmaceutical applications.

Suitable pharmaceutically-acceptable salts may take the form of base addition salts that may include, for example, metallic salts, e.g., alkali metal, alkaline earth metal and transition metal salts such as, for example, calcium, magnesium, potassium, sodium and zinc salts. The ammonium salt is a preferred salt.

Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. These salts may be prepared by conventional means from the subject compounds by reaction with the appropriate base.

The compounds used in the methods of the present invention may be administered by any route, including oral and parenteral administration. Parenteral administration includes, for example, intravenous, intramuscular, intraarterial, intraperitoneal, intranasal, rectal, intravesical, intradermal, topical or subcutaneous administration. Also contemplated within the scope of the invention is the instillation of drug in the body of the patient in a controlled formulation, with systemic or local release of the drug to occur at a later time. For example, the drug may be localized in a depot for controlled release to the circulation, or for release to a local site of tumor growth.

The specific dose of compound to obtain therapeutic benefit for treatment of a proliferative disorder will, of course, be determined by the particular circumstances of the individual patient including, the size, weight, age and sex of the patient, the stage of the disease, the aggressiveness of the disease, and the route of administration of the compound.

For example, a daily dosage of from about 0.01 to about 50 mg/kg/day may be utilized, or from about 1 to about 40 mg/kg/day, or from about 3 to about 30 mg/kg/day. Higher or lower doses are also contemplated. A therapeutically effective amount may also be estimated on the basis of the studies hereinafter described.

The daily dose of the compound may be given in a single dose, or may be divided, for example into two, three, or four doses, equal or unequal, but preferably equal, that comprise the daily dose. When given intravenously, such doses may be given as a bolus dose injected over, for example, about 1 to about 4 hours.

N⁶-benzyladenosine-5′-monophosphate and pharmaceutically acceptable salts thereof may be administered in the form of a pharmaceutical composition, in combination with a pharmaceutically acceptable carrier. The active ingredient in such formulations may comprise from 0.1 to 99.99 weight percent. By “pharmaceutically acceptable carrier” is meant any carrier, diluent or excipient which is compatible with the other ingredients of the formulation and not deleterious to the recipient.

The active agent is preferably administered with a pharmaceutically acceptable carrier selected on the basis of the selected route of administration and standard pharmaceutical practice. The active agent may be formulated into dosage forms according to standard practices in the field of pharmaceutical preparations. See Alphonso Gennaro, ed., Remington's Pharmaceutical Sciences, 18th Ed., (1990) Mack Publishing Co., Easton, Pa. Suitable dosage forms may comprise, for example, tablets, capsules, solutions, parenteral solutions, troches, suppositories, or suspensions.

For parenteral administration, the active agent may be mixed with a suitable carrier or diluent such as water, an oil (particularly a vegetable oil), ethanol, saline solution, aqueous dextrose (glucose) and related sugar solutions, glycerol, or a glycol such as propylene glycol or polyethylene glycol. Solutions for parenteral administration preferably contain a water soluble salt of the active agent. Stabilizing agents, antioxidant agents and preservatives may also be added. Suitable antioxidant agents include sulfite, ascorbic acid, citric acid and its salts, and sodium EDTA. Suitable preservatives include benzalkonium chloride, methyl- or propyl-paraben, and chlorbutanol. The composition for parenteral administration may take the form of an aqueous or nonaqueous solution, dispersion, suspension or emulsion.

For oral administration, the active agent may be combined with one or more solid inactive ingredients for the preparation of tablets, capsules, pills, powders, granules or other suitable oral dosage forms. For example, the active agent may be combined with at least one excipient such as fillers, binders, humectants, disintegrating agents, solution retarders, absorption accelerators, wetting agents absorbents or lubricating agents. According to one tablet embodiment, the active agent may be combined with carboxymethylcellulose calcium, magnesium stearate, mannitol and starch, and then formed into tablets by conventional tableting methods.

The pharmaceutical composition is preferably in unit dosage form. In such form the preparation is divided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compounds used in the methods of the present invention may be administered according to the invention in combination with one or more other active agents, for example, a least one other anti-proliferative compound, or drug to control side-effects, for example anti-emetic agents. The further active agent may comprise, for example a chemotherapeutic agent effective against prostate cancer. Such other agents for the treatment of prostate cancer include, for example, docetaxel, mitoxantrone, estramustine, doxorubicin, etoposide, vinblastine, paclitaxel, carboplatin, and vinorelbine.

In one embodiment of the invention, N⁶-benzyladenosine-5′-monophosphate, or pharmaceutically acceptable salt thereof, may be used to sensitize prostate cancer to radiation treatment and/or chemotherapy, e.g., docetaxel chemotherapy.

Radiation therapy uses high-energy rays or particles to kill cancer cells. The radiation treatment may comprise, for example, brachytherapy, i.e., implantation radiotherapy, or external beam radiation. Brachytherapy uses small radioactive pellets, or “seeds” implant radiation therapy or external-beam radiation therapy. Methods of external beam radiation and brachytherapy are well-known to those skilled in the art.

In one embodiment of the invention, the combination of N⁶-benzyladenosine-5′-monophosphate or pharmaceutically acceptable salt thereof, and the other anticancer agent, particularly an anticancer agent effective against prostate cancer, are co-formulated and used as part of a single pharmaceutical composition or dosage form. The compositions according to this embodiment of the invention comprise, as a primary agent, N⁶-benzyladenosine-5′-monophosphate or pharmaceutically acceptable salt thereof, and at least one other chemotherapeutic agent, in combination with a pharmaceutically acceptable carrier. In such compositions, the primary agent, and the second chemotherapeutic agent, may together comprise from 0.1 to 99.99 weight percent of the total composition. The compositions may be administered by any route and according to any schedule which is sufficient to bring about the desired antiproliferative effect in the patient.

According to other embodiments of the invention, the combination of the primary anticancer agent and the at least one other chemotherapeutic agent, may be formulated and administered as two or more separate compositions, at least one of which comprises the primary anticancer agent, and the other comprises the other chemotherapeutic agent. The separate compositions may be administered by the same or different routes, administered at the same time or different times, and administered according to the same schedule or on different schedules, provided the dosing regimen is sufficient to bring about the desired antiproliferative effect in the patient. When the drugs are administered in serial fashion, it may prove practical to intercalate administration of the two drugs, wherein a time interval, for example a 0.1 to 48 hour period, separates administration of the two drugs.

Prostate cancer cells, like certain normal prostate epithelial cells, can chronically depend on a critical level of androgenic stimulation for their net continuous growth and survival. Almost all prostate carcinomas are originally androgen-dependent. Androgen ablation has been used as a standard systemic therapy for metastatic prostate cancer. Androgen ablation is a type of therapy where the purpose is to remove or reduce the amount of androgen in a subject. Androgen ablation techniques for ablating serum androgens include, for example, androgen ablation by drug treatment, i.e., (i) treatment with luteinizing hormone releasing hormone (LHRH) agonists, e.g., goserelin or leuprolide, (ii) treatment with an anti-androgen such as flutamide or bicalutamide; or (iii) treatment with an agent that suppresses local synthesis of androgenic steroids in the prostate, e.g., the agent ketoconazole or abiraterone. Androgen ablation therapy can include a combination drug treatment including combining one or more drugs from two or three of the aforementioned categories. For example, at least one LHRH agonist may be combined with at least one anti-androgen and/or at least one inhbitor of prostate synthesis of androgenic steroids. Androgen ablation may also include castration in the form of surgical castration (orchiectomy, i.e., surgical removal of testes) or chemical castration. Androgen ablation therapy may include a combination of the drug-based therapy, described above, and castration.

Stat5a/b has been shown to synergize with androgen receptor (AR) in prostate cancer cells (Tan et al., Cancer Res 2008, 68:236-248). Specifically, active Stat5a/b increases transcriptional activity of AR, and AR, in turn, increases transcriptional activity of Stat5a/b. Liganded AR and active Stat5a/b physically interact in prostate cancer cells and, importantly, enhance nuclear localization of each other. This synergy between AR and the prolactin signaling protein Stat5a/b in human prostate cancer cells provides a further target for therapeutic intervention in the treatment of prostate cancer.

Accordingly, inhibition of Stat5a/b activity achieved with administration of one N⁶-benzyladenosine-5′-monophosphate or pharmaceutically acceptable salt thereof as a primary anticancer agent, when coupled with androgen ablation therapy, may lead to enhanced and synergistic inhibition of prostate cancer cell growth. Thus, according to another embodiment of the invention, the primary anticancer agent is administered in conjunction with an androgen ablation therapy for treatment of prostate cancer. The androgen ablation therapy may comprise drug-based androgen ablation such as treatment with an LHRH agonist and/or anti-androgen, castration, or both. Where drug-based androgen ablation is employed, the primary anticancer agent may be combined with the androgen ablation agent in a single composition or dosage form, separated in two compositions or dosage forms, administered by the same or separate routes, and administered simultaneously or at different times.

The practice of the invention is illustrated by the following non-limiting examples.

### EXAMPLES

Cells used in the following procedures were cultured according to the following conditions. Human prostate cancer cell lines PC3, DU145, CWR22Rv1 and LNCaP were cultured in RPMI 1640 containing penicillin/streptomycin and 10% fetal bovine serum (FBS), 0.5 nmol/L of dihydroestosterone (DHT) was additionally supplemented for LNCaP cells. African green monkey kidney cells COS-7 from ATCC were grown in DMEM (Invitrogen) supplemented with 10% FBS and penicillin/streptomycin. Cells were maintained in a 37° C. humidified incubator with a mixture of 95% air and 5% CO₂.

The following is the structure of the compound designated as “C5”, used as a control in certain of the following examples:

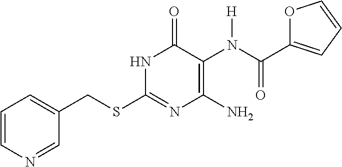 [SMILES: Nc1nc(SCc2cccnc2)[nH]c(=O)c1NC(=O)c1ccco1]

### Example 1

### N⁶-Benzyladenosine-5′-Monophosphate Blockade of Stat5a and Stat5b Transcriptional Activity

Cells of the human prostate cell line PC-3 were plated into 96-well plate at the density of 2×10⁵ per well. After 24 hours of plating, cells were transiently co-transfected using FuGENE6 (Roche) with 0.25 μg of each of pStat5a or pStat5b, pPrlR (prolactin receptor) plasmids, 0.5 μg of pBeta-casein-luc and 0.025 μg of pRL-TK (Renilla luciferase) plasmids as an internal control. After another 24 hours of transfection, the cells were starved in serum-free medium for 8 hours, pretreated with N⁶-benzyladenosine-5′-monophosphate (“N6BAP”), or one of five control compounds (designated “C3” through “C7”) of similar molecular weight but unrelated chemical structure to N6BAP), for 1 hour, and then stimulated with 10 nM human prolactin (hPrl) in the serum-free medium for additional 16 hours. The lysates were assayed for firefly and Renilla luciferase activities using the Dual-Luciferase reporter assay system (Promega). Three independent experiments were carried out in triplicate. The firefly luciferase activity was normalized to the Renilla luciferase activity of the same sample, and the mean was calculated from the parallels. From the mean values of each independent run, the overall mean and its standard deviation (S.D.) were calculated.

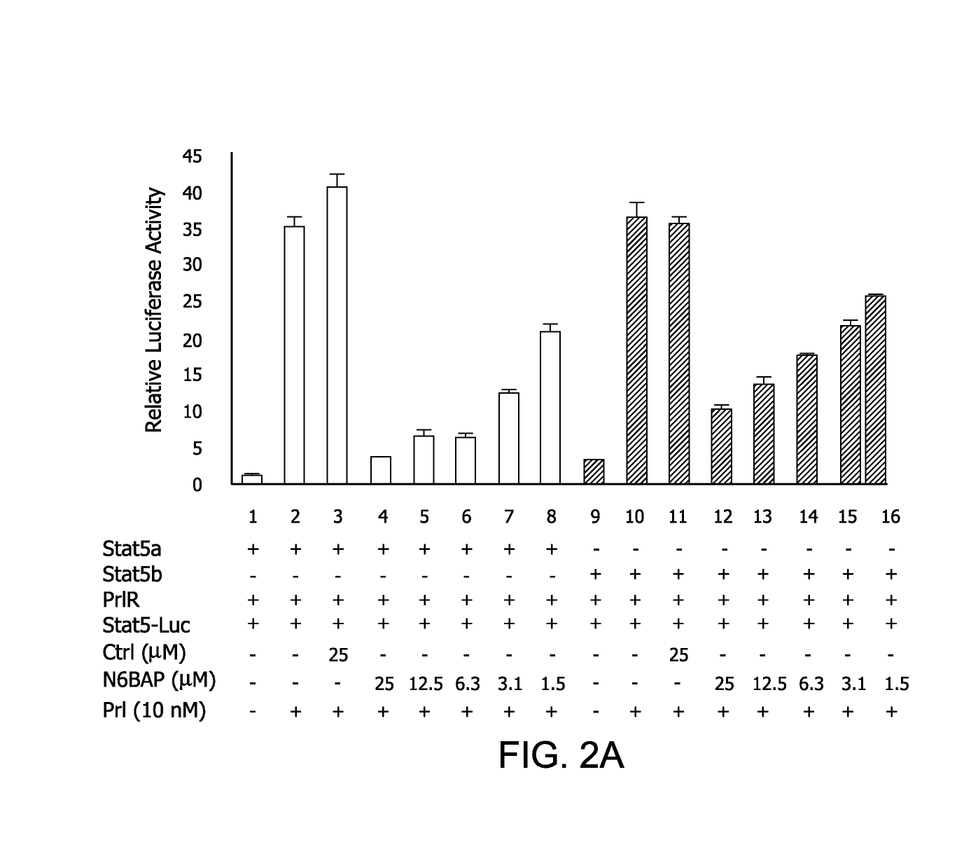

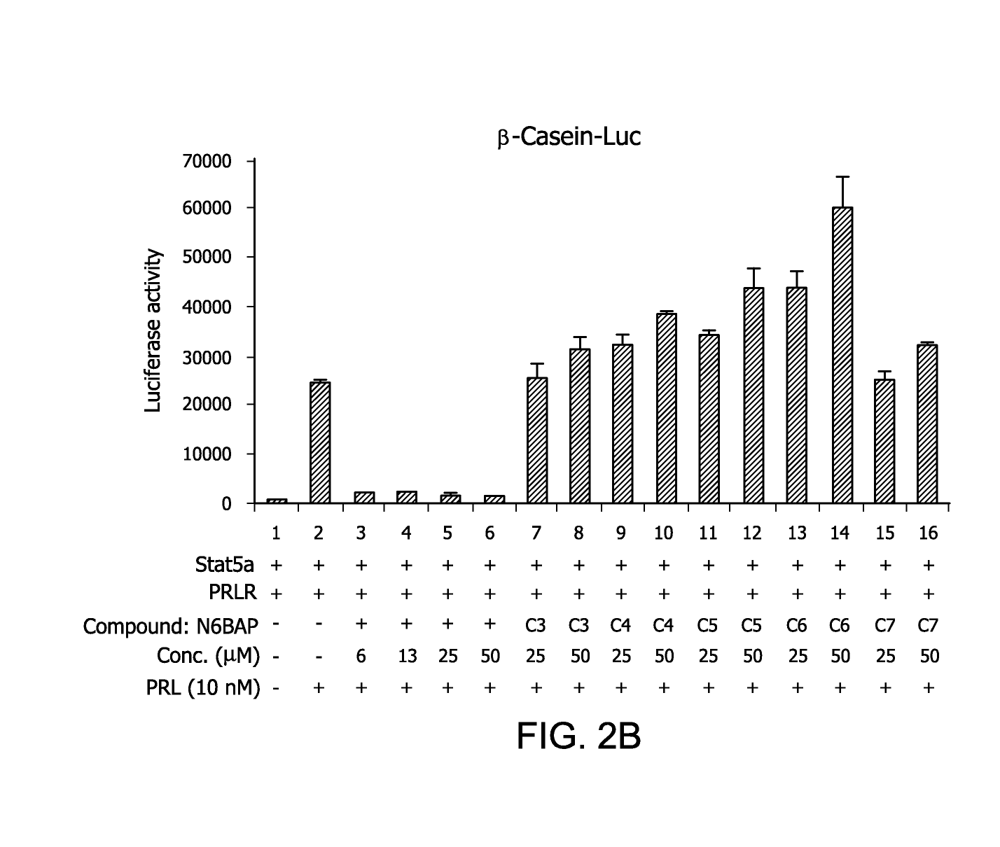

The results are shown in FIGS. 2A and 2B. According to FIG. 2A, N6BAP effectively blocks the transcriptional activity of Stat5a and Stat5b in the PC-3 human prostate cancer cells. According to FIG. 2B, none of the five control compounds of molecular weight similar to N6BAP, but unrelated in structure (“C3” through “C7”), had any effect on the transcriptional activity of Stat5a or Stat5b.

### Example 2

### N⁶-Benzyladenosine-5′-Monophosphate Inhibition of Stat5 Dimerization Assay

Dimerization of Stat5a/b molecules is required for transcriptional activity of Stat5a/b and its biological effects. The following study demonstrates that N6BAP blocks dimerization of Stat5a/b in human prostate cancer cells.

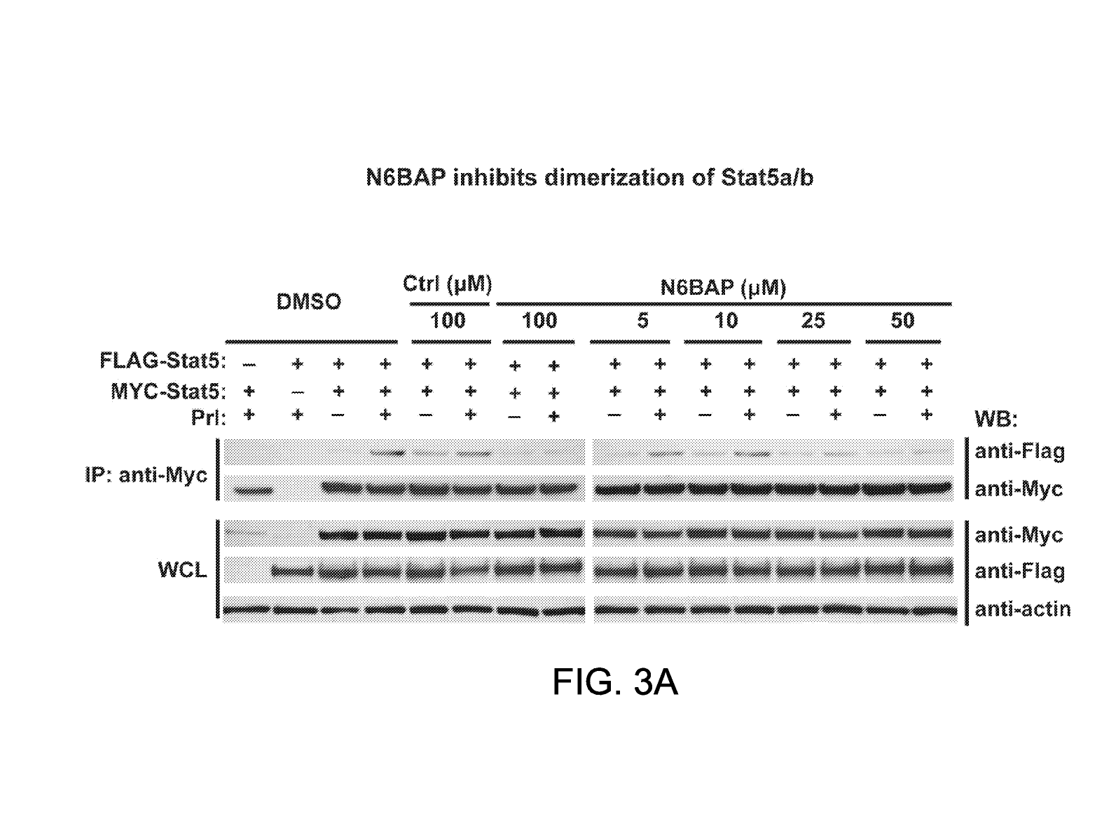

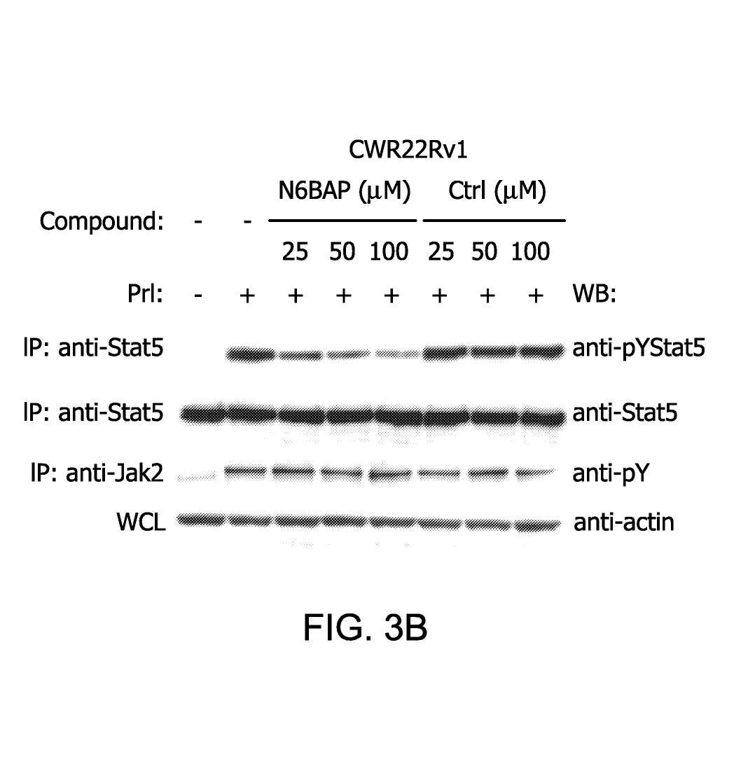

FLAG-tagged Stat5a and MYC-tagged Stat5a were generated as follows. Plasmid pCMV3-FLAG-Stat5a, pCMV3-MYC-Stat5a and pPrlR were co-transfected using FuGENE6 (Roche) into PC-3 cells (2 μg of each plasmid per 1×10⁷ cells). The cells were starved for 20 hours, pretreated with N6BAP for 2 hours, then stimulated with hPrl (10 nM) in RPMI 1640 without serum for 30 minutes. The cell lysates were immunoprecipitated with 25 μl anti-FLAG M2 polyclonal affinity gel (2 μg/ml, Sigma), anti-MYC pAb (1 μg/ml, Upstate) or normal rabbit serum (NRS). The primary antibodies were used in the immunoblottings at the following concentrations: anti-FLAG pAb (1:1000; Stratagene), anti-MYC mAb (1:1000; Sigma), anti-Stat5a/b mAb (1:250) (Transduction Laboratories) detected by horseradish peroxidase-conjugated secondary antibodies. The results are shown in FIG. 3A.

Lane 1 of FIG. 3A demonstrates cells transfected only with MYC-tagged Stat5a (the third panel from the bottom). Lane 2 demonstrates cells transfected only with FLAG-tagged Stat5a (the second panel from the bottom). Lanes 3-16 demonstrate cells transfected with both FLAG-tagged Stat5a and MYC-tagged-Stat5a (the second and third panels from the bottom, respectively). Lanes 1, 2, 4, 6, 8, 10, 12, 14 and 16 represent cells stimulated with human prolactin (Prl) for 30 minutes which induces dimerization of Stat5. Two hours prior to Prl-stimulation, the cells had been treated with DMSO (lanes 1-4), the control compound C5 (lanes 5 and 6) or N6BAP (lanes 7-16) to test whether the Stat5a/b-inhibitor compound, N6BAP, would be able to inhibit dimerization of Stat5. When MYC-tagged Stat5a was immunoprecipitated from the cells (the second panel from the top) and immunoblotted with anti-FLAG pAb (top panel) the control compound did not inhibit dimerization of Stat5a (lane 6). In contrast, N6BAP effectively inhibited Stat5a dimerization (lanes 8, 14, 16) (=very weak band in Prl-stimulated cells). It should be noted the drug effect in this study was not due to cell apoptosis induction, as the treatment time was only 2 hours. Treatment of at least 48 hours is required for the compound to induce prostate cancer cell apoptosis.

### Example 3

### N⁶-Benzyladenosine-5′-Monophosphate Inhibition of Stat5 Phosphorylation in Prostate Cells

CWR22Rv1 cells were starved for 24 h in serum-free medium, treated N6BAP at 25, 50 and 100 μM concentrations for 2 h, followed by stimulation with 10 nM Prl for 30 minutes. Stat5a and Jak2 were immunoprecipitated with anti-Stat5a or anti-Jak2 pAbs. Immunoprecipitates of CWR22Rv1 cells were blotted with anti-PYStat5, anti-Stat5a/b mAb or anti-PY mAb (for Jak2). Whole cell lysates were immunoblotted with anti-actin pAb for loading control. Because SH2-domain of Stat5 mediates the recruitment of Stat5 to the Prl-receptor-Jak2-complex and Stat5 phosphorylation, N6BAP inhibits Prl-induced phosphorylation of Stat5.

The protocol in more detail comprised the following. Cell pellets were solubilized in lysis buffer [10 mM Tris-HCl (pH 7.6), 5 mM EDTA, 50 mM sodium chloride, 30 mM sodium pyrophosphate, 50 mM sodium fluoride, 1 mM sodium orthovanadate, 1% Triton X-100, 1 mM phenylmethylsulfonyl fluoride, 5 μg/mlaprotinin, 1 μg/ml pepstatin A, and 2 μg/ml leupeptin], rotated end-over-end at 4° C. for 60 min, and insoluble material was pelleted at 12,000×g for 30 min at 4° C. The protein concentrations of clarified cell lysates were determined by simplified Bradford method (Bio-Rad Laboratories, Hercules, Calif.). Stat5a, Stat5b and Jak2 were immunoprecipitated from whole cell lysates with anti-Stat5a, anti-Stat5b (4 μl/ml; Millipore, Billerica, Mass.) or anti-Jak2 (Millipore) pAbs. Antibodies were captured by incubation for 60 min with protein A-Sepharose beads (Amersham Pharmacia Biotech, Piscataway, N.J.). For Western blotting, the primary antibodies were used at the following concentrations: anti-phosphotyrosine-Stat5a/b (Y694/Y699) mAb (1 μg/ml, Advantex BioReagents, Conroe, Tex.), anti-Stat5ab mAb (1:250; BD Biosciences, San Jose, Calif.). For immunoblotting of phosphotyrosine-Jak2, we used anti-phosphotyrosine mAb (Millipore). Other antibodies for Western blotting were anti-β-actin pAb (1:4,000; Sigma.

The results in FIG. 3B show that N6BAP inhibits Stat5 phosphorylation in the human prostate cell line CWR22Rv1.

### Example 4

### N⁶-Benzyladenosine-5′-Monophosphate Inhibition of Stat5a Translocation to the Nucleus in Human Prostate Cells

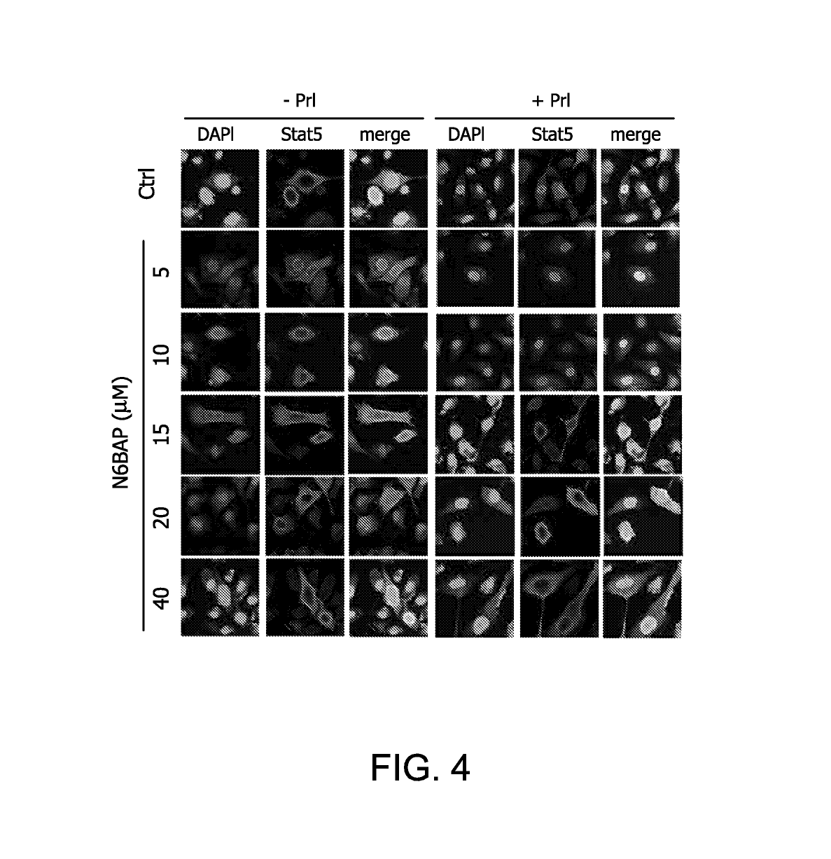

PC3 cells were infected with Ade-Stat5a and Ade-PrlR at MOI 5 for each adenovirus. Cells were pretreated with N6BAP or vehicle (DMSO) for 2 hours and then stimulated with 10 nmol/L Prl for 30 minutes. Cells were fixed with 100% methanol for 15 min and subsequently permeabilized with 0.1% Triton X-100 in PBS (pH 7.4). After blocking with 2% bovine serum albumin in PBS, cells were incubated with an antibody specific for Stat5a or Stat5b at 4° C. for 2 hours. Cells were then washed with PBS and incubated with FITC-conjugated secondary antibody (Jackson ImmunoResearch) at room temperature for 1 hour. The images were taken by using an inverted fluorescence microscopy (Carl Zeiss). The results are shown in FIG. 4. N6BAP blocks translocation of Stat5a to the nucleus of human prostate cancer cells after ligand (prolactin)-induced activation. It should be noted the drug effect in this study was not due to cell apoptosis induction, as the treatment time was only 2 hours. Treatment of at least 48 hours is required for the compound to induce prostate cancer cell apoptosis.

### Example 5

### N⁶-Benzyladenosine-5′-Monophosphate Inhibition of Stat5a Binding to DNA

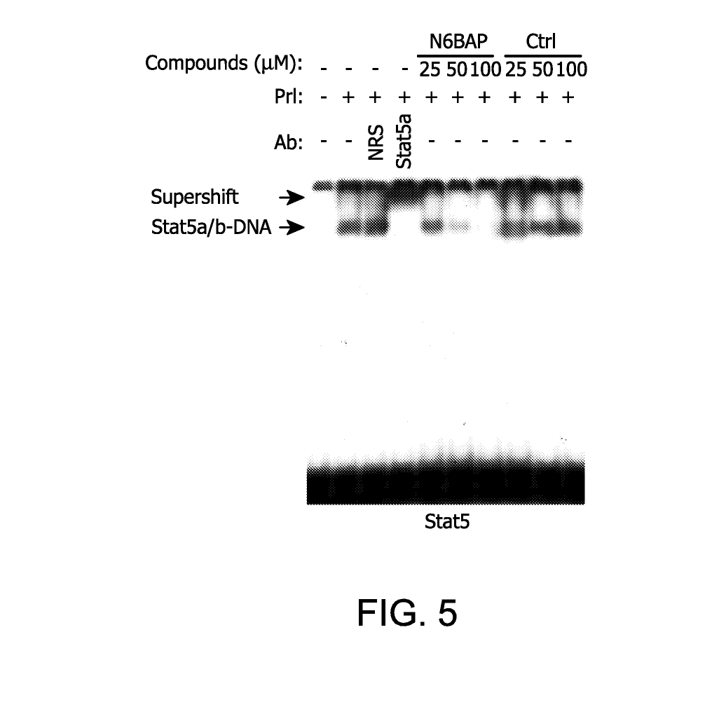

The following electrophoresis mobility shift assay (EMSA) was carried out to demonstrate that N6BAP inhibits binding of Stat5a/b to DNA after ligand induced activation. COS-7 cells were transfected with plasmids expressing Stat5a (pStat5a) and PrlR (pPrlR) using FuGENE6. After 24 hours, the cells were starved in serum-free medium for 16 hours and then pretreated with N6BAP or the control compound C5 for 2 hours and the stimulated with 10 nmol/L Prl for 30 minutes. Nuclear extracts were prepared and a gel EMSA was performed as previously described (Ahonen et al., Endocrinology 2002, 143:228-238; Nevalainen et al., Mol Endocrinol 2002, 16:1108-1124). Double stranded Stat5a/b-binding oligonucleotide probe (upper strand sequence: 5′-AGATTTCTAGGAATTCAATCC-3′) (Boucheron et al., J Biol Chem (1998), 273:33936-33941) were end-labeled with 50 μCi of γ-³²P-ATP (5000 ci/mMol) and incubated (1 ng per reaction) with 10 μl of nuclear extracts in the final volume of 20 μl of the binding mixture (50 mM TrisHCl, pH 7.4, 25 mM MgCl₂, 500 mM KCl, 5 mM DTT, 50% glycerol) and 1 μl of 1 mg/ml Poly d[(I:C)]. For a supershift control, the samples were preincubated with anti-Stat5a or anti-Stat5b antibody (Millipore) or NRS as indicated. Polyacrylamide gels (5%) containing 5% glycerol and 0.25×Tris-borate/EDTA were pre-run in 0.25×Tris-borate/EDTA buffer at 4-10° C. for 2-4 h at 300V. The gels were run at room temperature for 2.33 hours at 200 V, dried and exposed to x-ray films (X-Omat, Eastman Kodak Co.). The results are shown in FIG. 5. N6BAP inhibited binding of Stat5a/b to DNA after ligand-induced activation. It should be noted the drug effect in this study was not due to cell apoptosis induction, as the treatment time was only 2 hours. Treatment of at least 48 hours is required for the compound to induce prostate cancer cell apoptosis.

### Example 6

### N⁶-Benzyladenosine-5′-Monophosphate Downregulation of Expression of Stat5 Target Genes Cyclin D1 and Bcl-xL in Prostate Cancer Cells

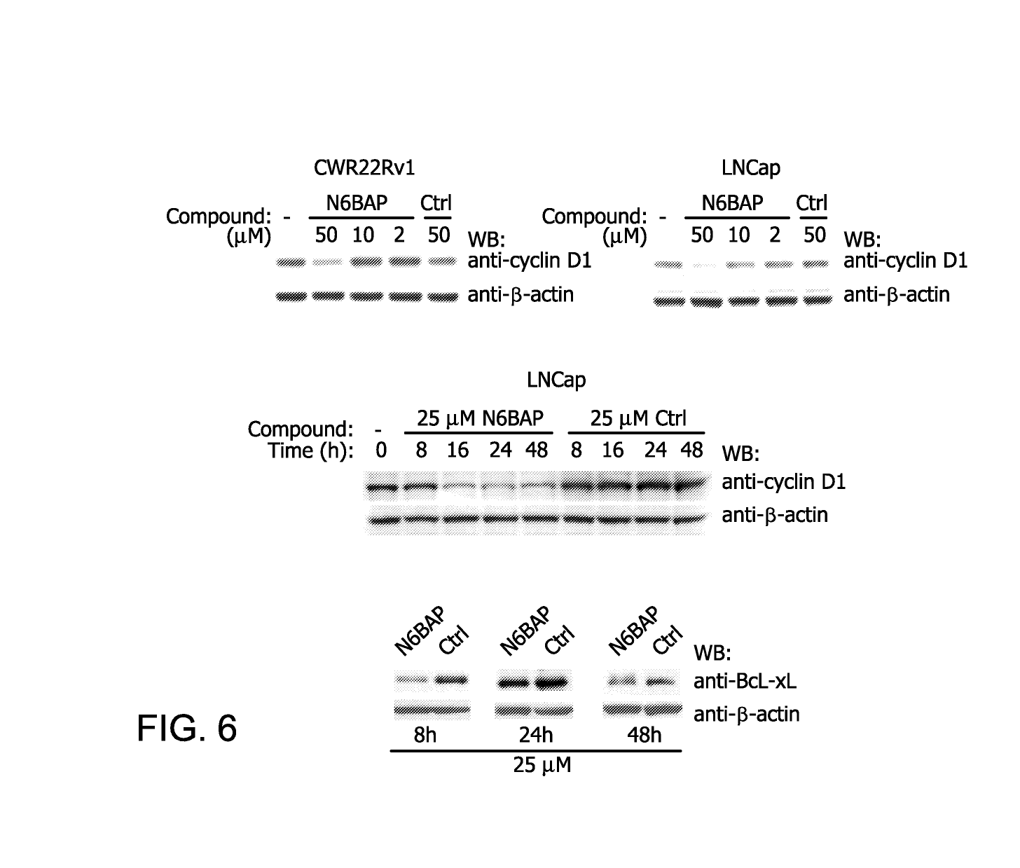

Exponentially growing CWR22Rv1 and LNCaP cells were treated with N6BAP or the control compound C5 (Ctrl) for 48 h at concentrations indicated in FIG. 6 and whole cell lysates were immunoblotted with anti-cyclinD1 mAb. Exponentially growing CWR22Rv1 and LNCaP cells were treated with N6BAP or control compound C5 (Ctrl) at 25 μM for the periods of time indicated in FIG. 6, and whole cell lysates were immunoblotted with anti-cyclinD1 mAb or anti-Bcl-X1 pAb. Cell pellets were solubilized in lysis buffer [10 mM Tris-HCl (pH 7.6), 5 mM EDTA, 50 mM sodium chloride, 30 mM sodium pyrophosphate, 50 mM sodium fluoride, 1 mM sodium orthovanadate, 1% Triton X-100, 1 mM phenylmethylsulfonyl fluoride, 5 μg/ml aprotinin, 1 μg/ml pepstatin A, and 2 leupeptin], rotated end-over-end at 4° C. for 60 min, and insoluble material was pelleted at 12,000×g for 30 min at 4° C. The protein concentrations of clarified cell lysates were determined by simplified Bradford method (Bio-Rad Laboratories, Hercules, Calif.). The following primary antibodies were used for Western blotting, at the following concentrations: anti-β-actin pAb (1:4,000; Sigma), anti-cyclin D1 mAb (1:1000, BD Biosciences) and Bcl-xL pAb (1:1000, Cell Signaling, Danvers, Mass.).

The results, shown in FIG. 6, demonstrate that N6BAP down-regulates expression of Stat5 target genes cyclin D1 and Bcl-xL in prostate cancer cells.

### Example 7

### N⁶-Benzyladenosine-5′-Monophosphate does not Inhibit Transcriptional Activity of Stat3 in LNCap Cells

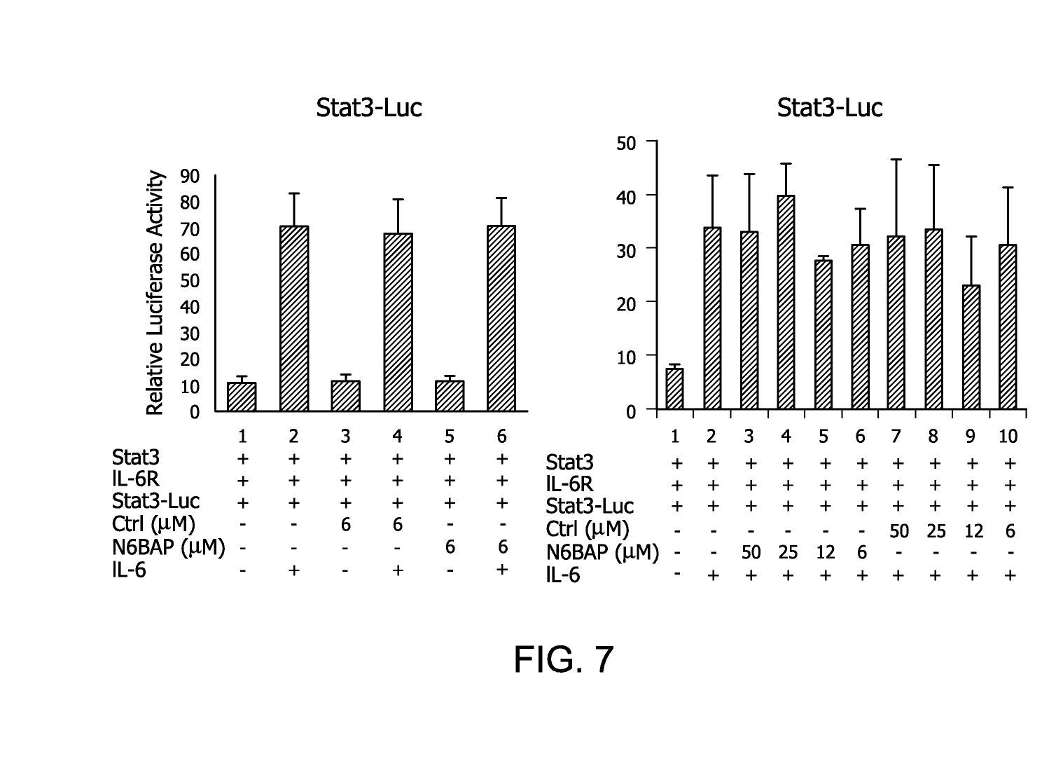

LNCaP prostate cancer cells were transiently co-transfected with pStat3, pIL-6-receptor (pIL-6R), pStat3-Luc and pRL-TK (Renilla luciferase). The cells were serum-starved for 20 h, pre-treated with N6BAP or the control compound C5 (Ctrl) at 6.0 μM for 2 h followed by stimulation with 50 ng/ml of IL-6 for 16 h. The relative luciferase activities were determined. The results, comprising the mean values of three independent experiments performed in triplicate, are shown in FIG. 7. S.E. values are indicated by bars.

The results demonstrate that the effect of N6BAP is specific to the SH2-domain of Stat5. The compound does not inhibit the transcriptional activity of Stat3 in prostate cancer cells.

### Example 8

### N⁶-Benzyladenosine-5′-Monophosphate is Specific for the SH2-Domain of Stat5 and does not Inhibit Nuclear Translocation of Stat3 in Prostate Cancer Cells

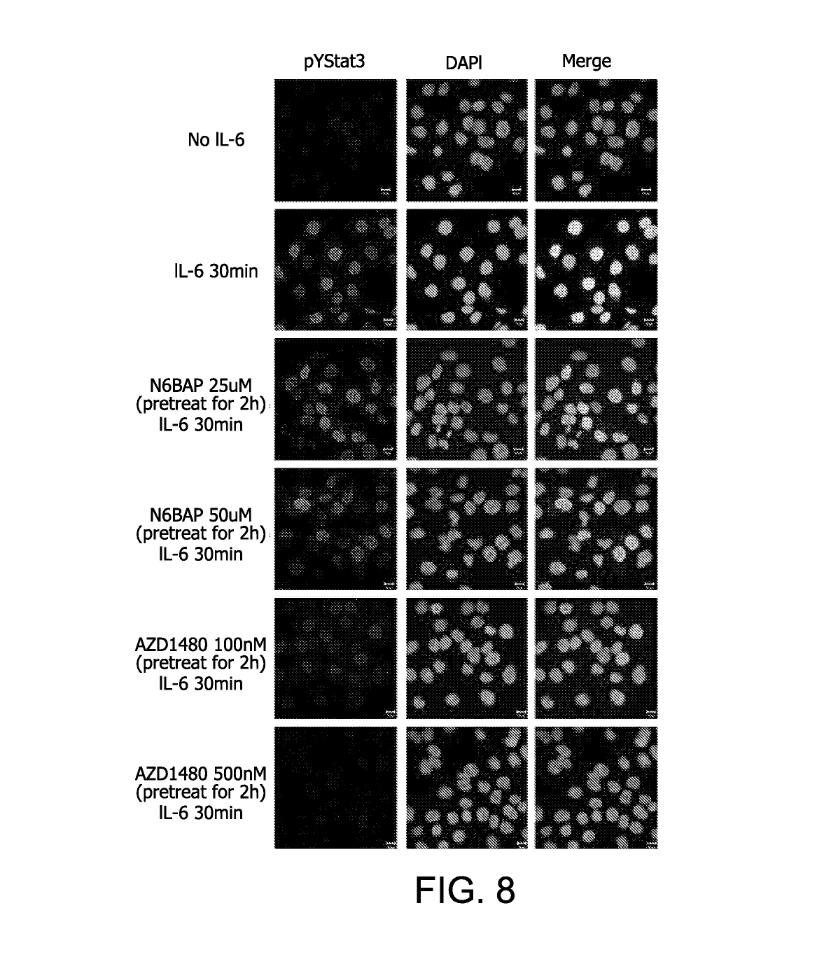

DU145 prostate cancer cells were pretreated with N6BAP or vehicle (DMSO) for 2 hours and then stimulated with 10 nmol/L IL-6 for 30 minutes. Cells were fixed with 100% methanol for 15 min and subsequently permeabilized with 0.1% Triton X-100 in PBS (pH 7.4). After blocking with 2% bovine serum albumin in PBS, cells were incubated with an antibody specific for Stat3 at 4° C. for 2 hours. Cells were then washed with PBS and incubated with FITC-conjugated secondary antibody (Jackson ImmunoResearch) at room temperature for 1 hour. The images were taken by using an inverted fluorescence microscopy (Carl Zeiss). The results are shown in FIG. 8. The results of these studies show that N6BAP did not block translocation of Stat3 to the nucleus of human prostate cancer cells after ligand (IL-6)-induced activation.

### Example 9

### N⁶-Benzyladenosine-5′-Monophosphate Induction of Human Prostate Cell Death

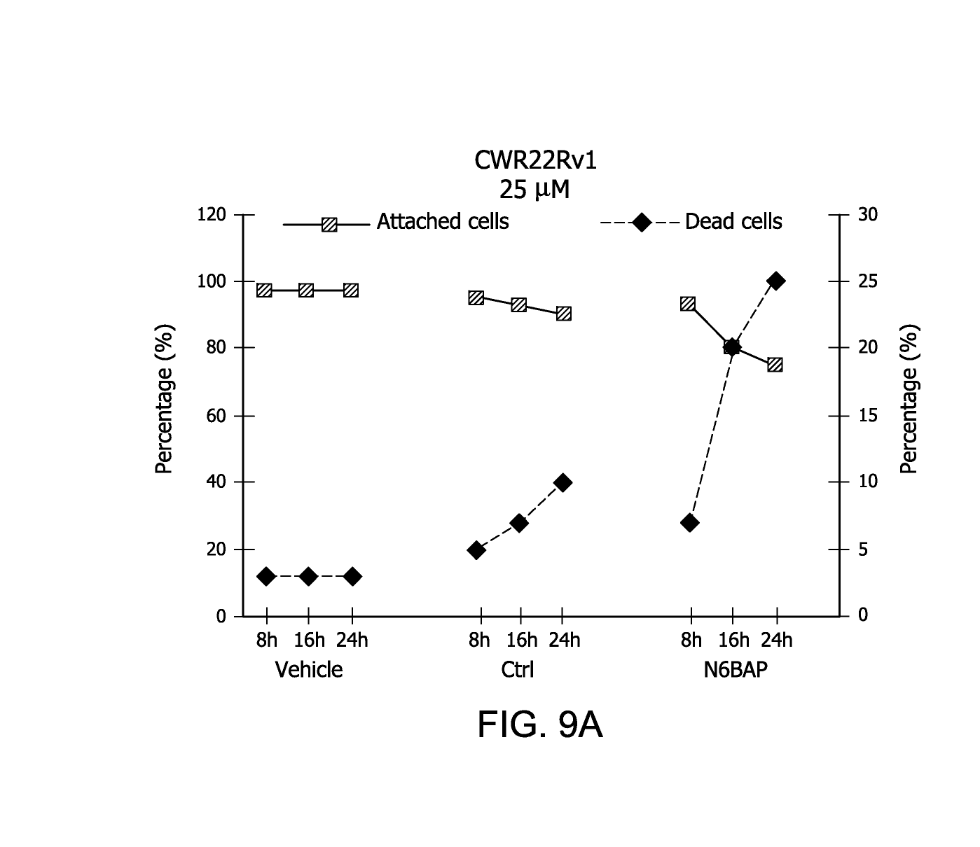

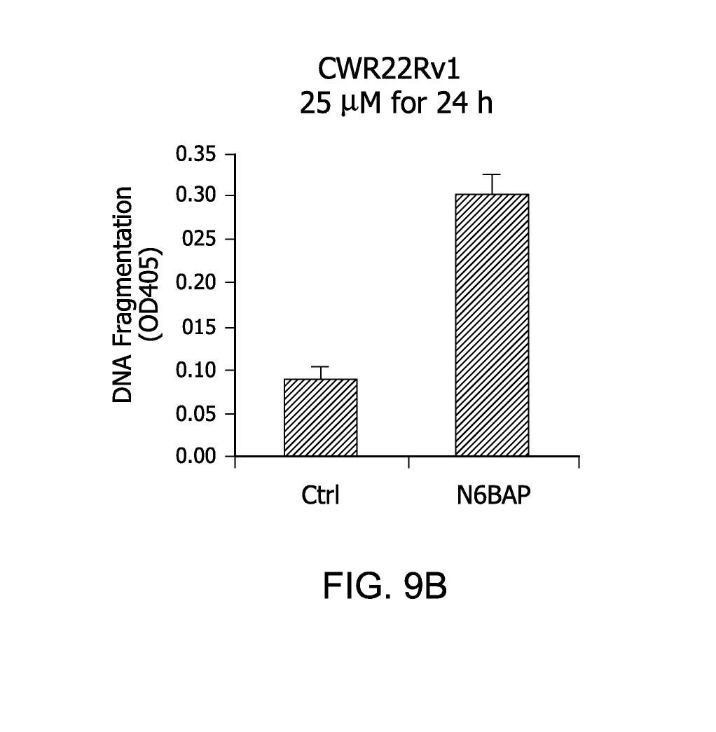

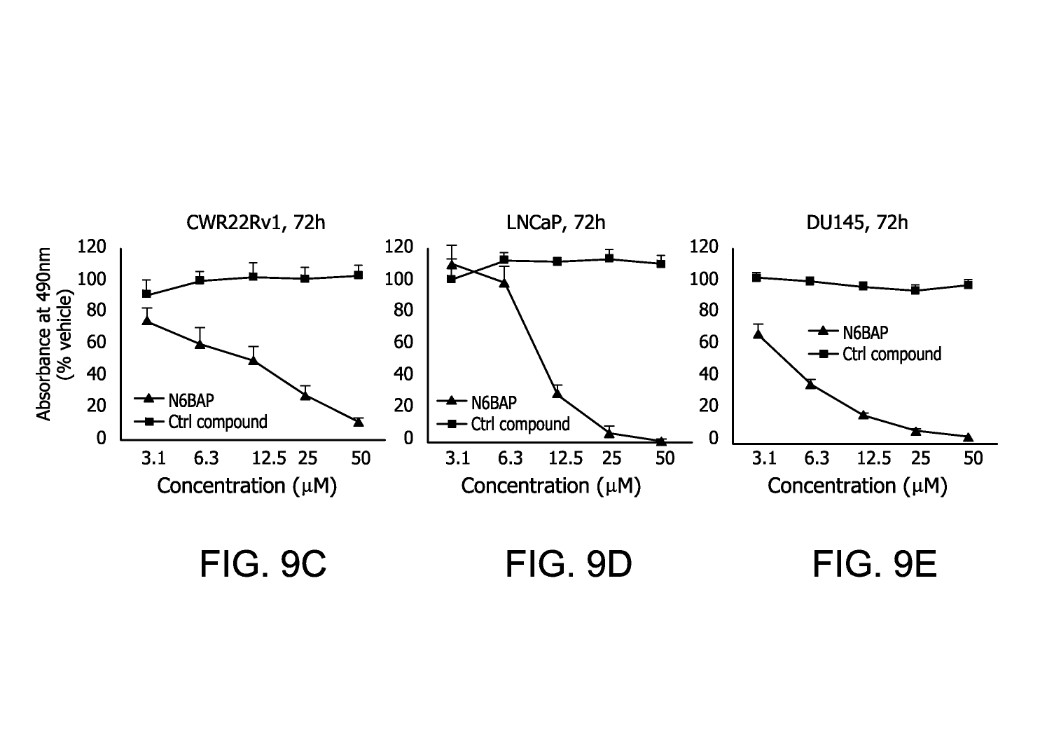

CWR22Rv1 prostate cancer cells were treated with N6BAP or the control compound C5 (Ctrl) at 25 μM concentration or vehicle for 24 h. Following trypan blue exclusion, the attached viable vs. de-attached dead cells were counted. The results are shown in FIG. 9A. In the parallel wells, nucleosomal DNA fragmentation indicating cell death due to apoptosis was demonstrated by nucleosomal ELISA. The results are shown in FIG. 9B. The bars indicate mean±SD of triplicate wells. In another assay, CWR22Rv1, LNCaP and DU145 cells were treated with N6BAP, the control compound C5 (Ctrl) or vehicle at indicated concentrations. The fraction of alive cells was determined by MTS (3-(4,5-dimethylthiazolyl-2)-2,5-diphenyl-tetrazolium bromide) metabolic activity assay (CellTiter 96® AQueous Assay kit). The results are shown in FIGS. 9C, 9D and 9E, respectively.

The results of the studies shown in FIGS. 9A-9E demonstrate that N6BAP induces apoptotic death of prostate cancer cells.

### Example 9a

### Comparative

### N⁶-Benzyladenosine-5′-Monophosphate does not Induce Cell Death in Certain Other Solid Tumor Cell Lines

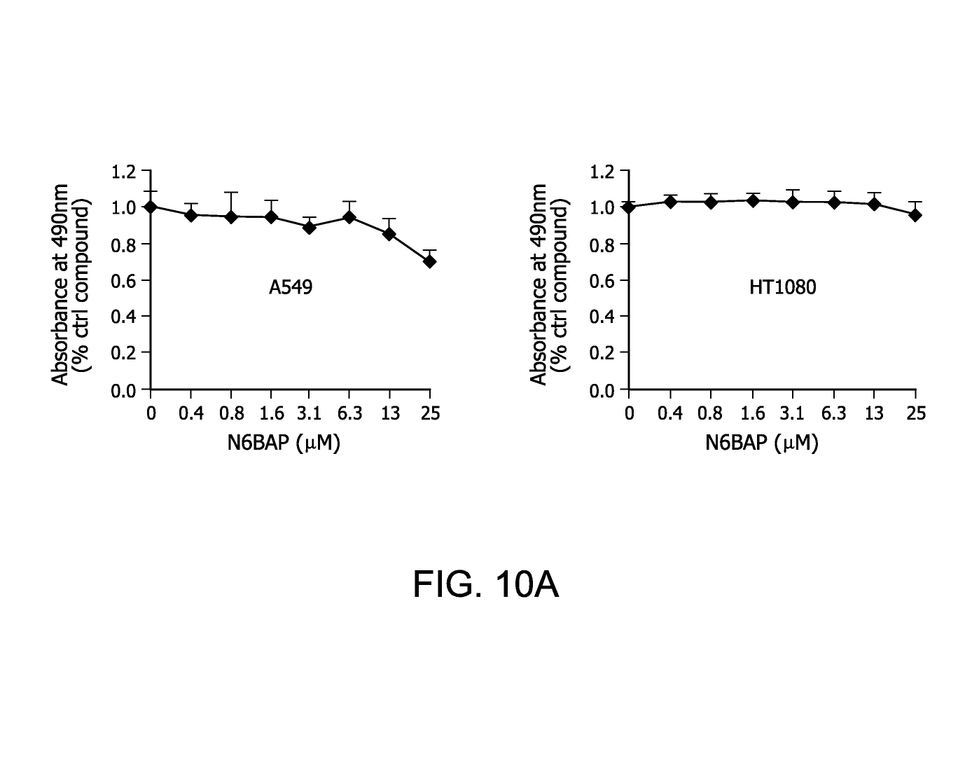

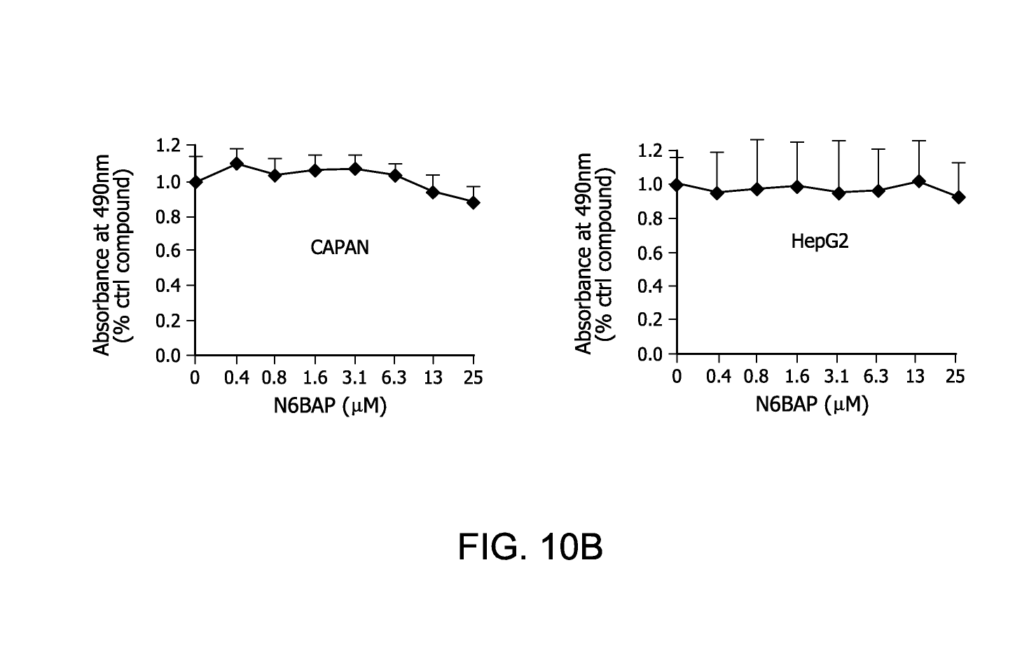

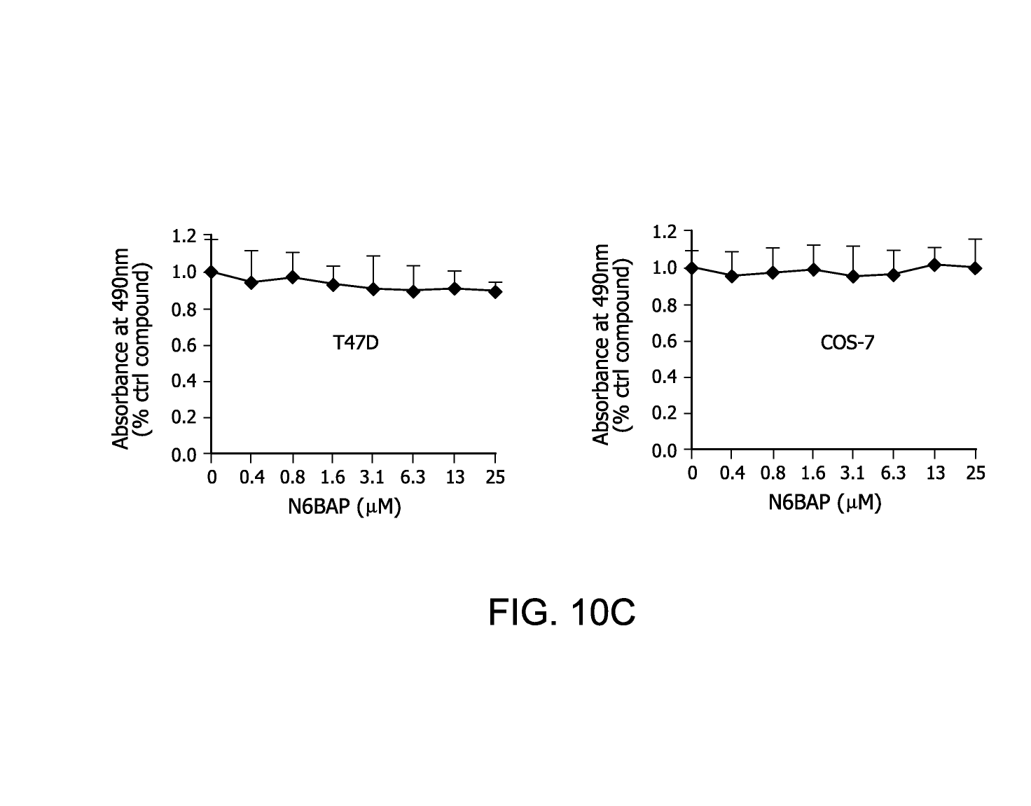

The procedure of Example 9 was followed, substituting cells of the following solid tumor cell lines for the cells treated in Example 9: A549 human lung cancer cells, CAPAN human pancreatic cancer cells, T47D human breast cancer cells, HCT116 human liver cancer cells, A2058 human melanoma and COS-7 monkey fibroblast cell lines. As shown in FIGS. 10A-10C, N6BAP did not affect the viability of any of these solid tumor cell lines.

### Example 10

### N⁶-Benzyladenosine-5′-Monophosphate Inhibits Human Prostate Tumor Xenograft Growth in Nude Mice

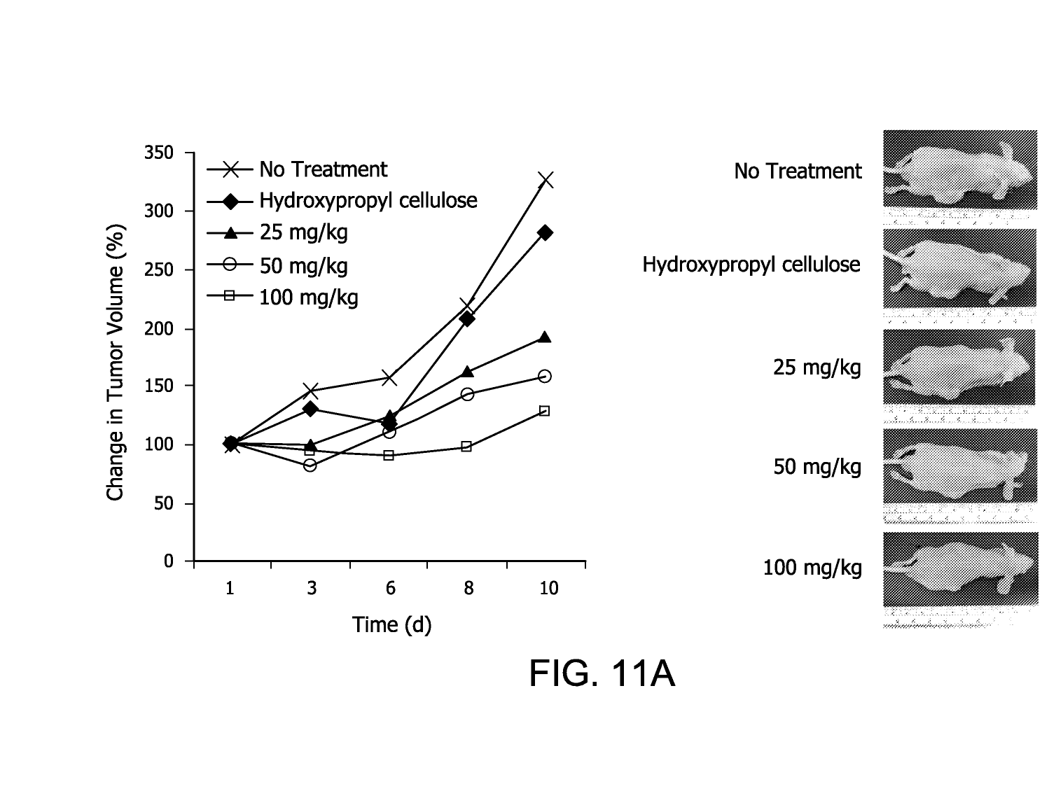

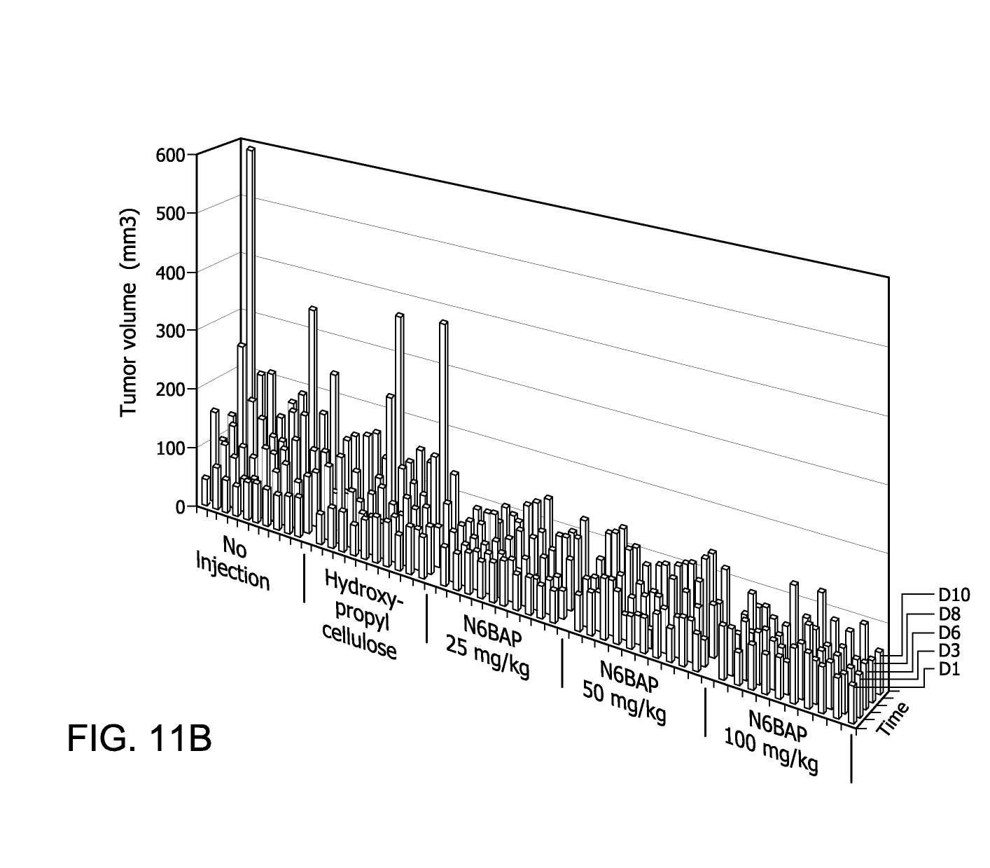

1.5×10⁷ CWR22Rv1 cells in 0.1 ml RPMI 1640 medium were mixed with 0.1 ml of Matrigel (BD Bioscience, Palo Alto, Calif.), and were inoculated subcutaneously (s.c.) into one flank per mouse. Established tumors were randomly distributed into five groups (ten mice for each group) with similar average size. N6BAP was dissolved in 0.3% hydroxycellulose (HPC). Mice were treated daily for 10 days by intraperitoneal injection with 0.2 ml of N6BAP at 25 mg/kg, 50 mg/kg or 100 mg/kg body weight, or with 0.2 ml 0.3% HPC solution, or no treatment for control. Tumor sizes were measured three times per week. Tumor volumes were calculated using the following formula: 3.14×length×width×depth/6. Tumor growth rates were calculated, and percent changes in tumor volume of each group were presented. The results are presented in FIG. 11A. N6BAP inhibited the growth of the xenograft tumor arising from the implantation of the human prostate cancer cell line CWR22Rv1. FIG. 11B shows tumor xenograft volumes for the individual tumors in all treatment groups which contributed to the data of FIG. 11A. Individual tumors are represented as columns.

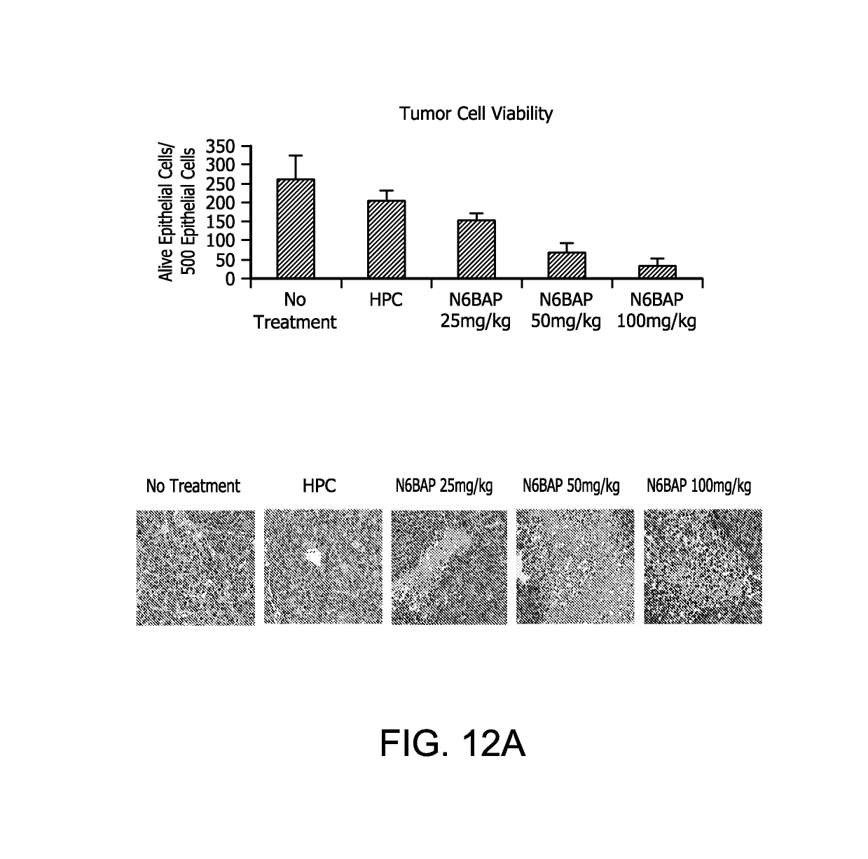

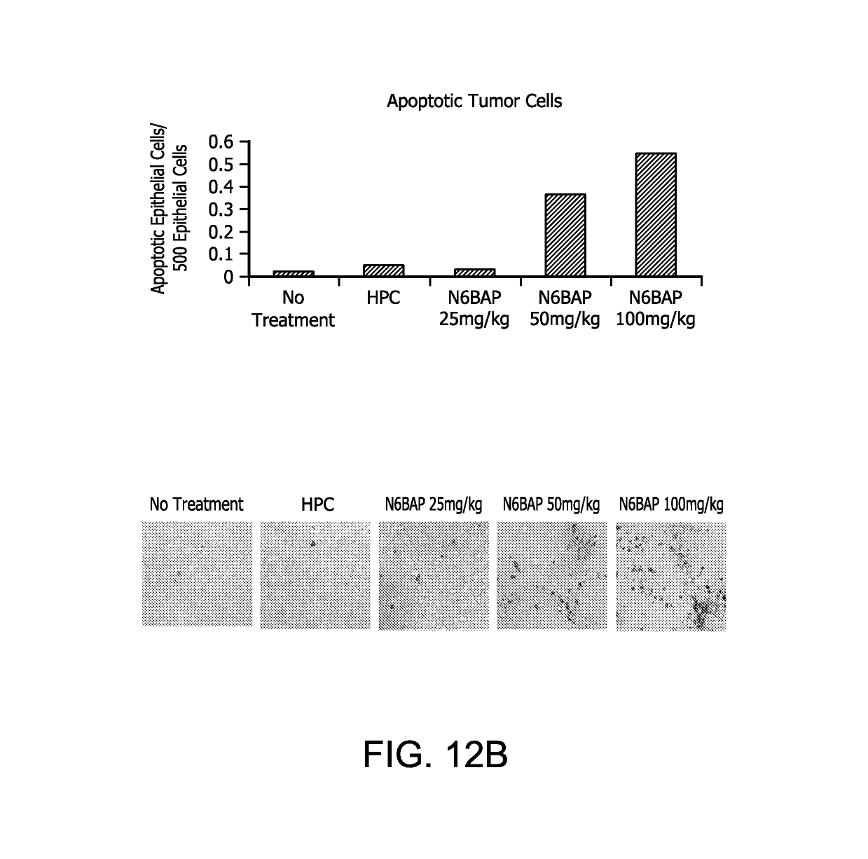

Tumor sections were hematoxylin-eosin stained to assess the loss of viable tumor cells. The results are shown in FIG. 12A, showing the loss of live epithelial cells with the viable N6BAP-treated CWR22Rv1 xenograft tumors vs. controls.

In situ end labeling of fragmented DNA was carried out according to the terminal deoxyribonucleotidyl transferse (TdT)-mediated biotin-16-dUTP nick-end labeling (TUNEL) assay to determine that cells within the CWR22Rv1 tumors were undergoing apoptosis. The assay was performed according to the In situ Cell Death Detection Kit from Roche Applied Science. Briefly, rehydrated deparaffinated tissue sections from the xenograft tumors were treated by Proteinase K followed by 3% H₂O₂ at room temperature. Fluorescein-labeled deoxynucleotides were catalytically added to 3′-OH ends of double- or single-stranded DNA by terminal deoxynucleotidyl transferase. Nucleotides incorporated into fragmented DNA were detected after incubation with anti-fluorescein antibody conjugated with peroxidase followed by visualization with 3,3-diaminobenzidine as a chromogen and methyl green as a counterstain. Three microscopic views of each tumor (ten tumors per each treatment group) were photographed at 20× magnification. Cell viability indexes were determined by counting alive cells per total number of cells per view established in the no treatment group. TUNEL indexes were determined by counting epithelial cells with TUNEL-positive cells per total number of epithelial cells per view. The counts were averaged within the tumors in a given treatment group.

The results are shown in FIG. 12B. N6BAP increased apoptotic epithelial cells within the CWR22Rv1 prostate cancer xenograft tumors grown in nude mice.

### Example 11

### N⁶-Benzyladenosine-5′-Monophosphate Effect on Nuclear Stat5 Expression and Nuclear Stat3 Expression in Prostate Tumor Xenograft Growth in Nude

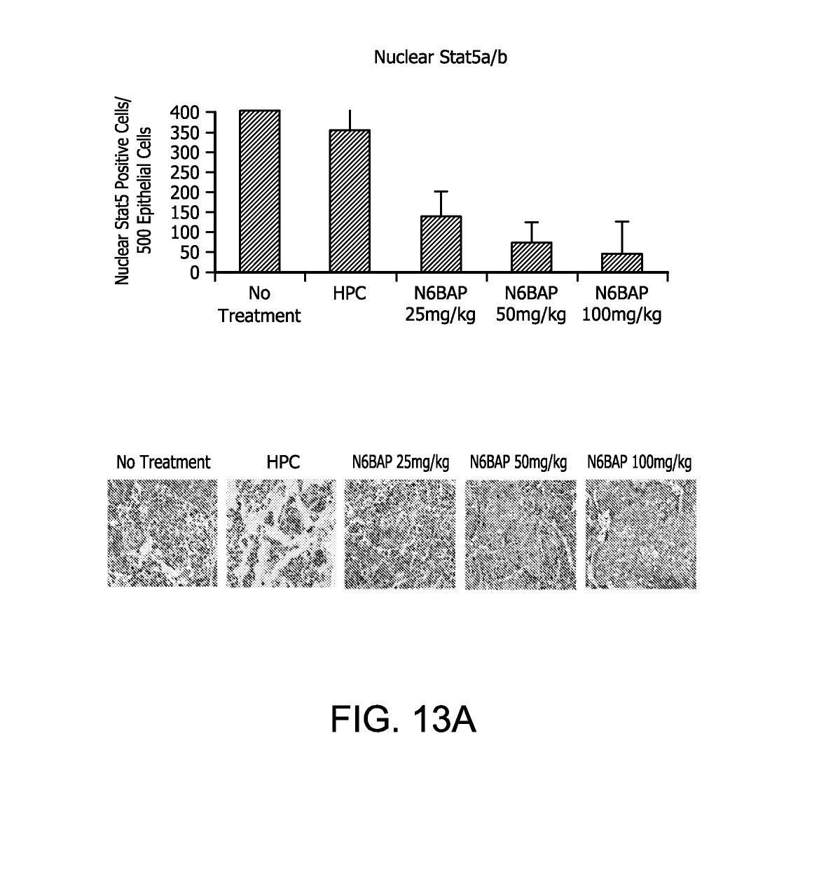

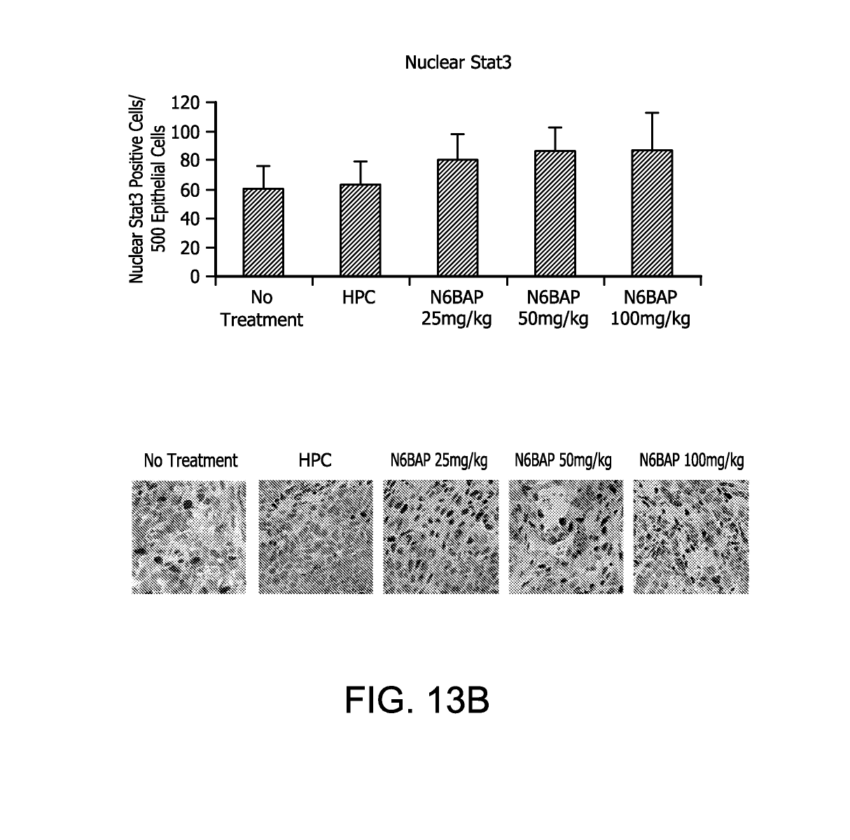

Nuclear active Stat5a/b or nuclear active Stat3 were analyzed by immunostaining with an anti-Stat5a/b or anti-Stat3 antibody, respectively, and biotin-streptavidin amplified peroxidase antiperoxidase immunodetection of paraffin-embedded sections of the prostate xenograft tumors of Example 10. Anti-Stat5a/b (mAb) (55 ng/ml) (Santa Cruz Biotechnology) was used as the primary antibody and antigen-antibody complexes were detected by appropriate biotinylated goat secondary antibodies (Biogenex Laboratories) followed by streptavidin-horseradish-peroxidase complex, and 3,3′-diaminobenzidine as chromogen and Mayer hematoxylin as counterstain. Three microscopic views of each tumor (ten tumors per each treatment group) were photographed at 20× magnification. Nuclear Stat5 and Stat3 indexes were determined by counting epithelial cells with nuclear Stat5a/b immunostaining-positive cells per total number of epithelial cells per view. The counts were averaged within the tumors in a given treatment group. The results, shown in FIGS. 13A and 13B, demonstrate that N6BAP inhibits nuclear Stat5a/b expression in CWR22Rv1 prostate cancer xenograft tumors (FIG. 13A) without affecting nuclear Stat3 (FIG. 13B).

### Example 12

### N6-Benzyladenosine-5′-Monophosphate Induces Death of Prostate Cancer Acinar Epithelium in Clinical Prostate Cancers Tested Ex Vivo in Explant Organ Cultures

For organ cultures, prostate cancer specimens were obtained during surgery from eight patients with localized or locally advanced prostate cancer undergoing radical prostatectomy and bilateral iliac lymphadenectomy. The specimens are identified in Table 1.

TABLE 1
Prostate cancers cultured ex vivo in explant organ cultures.

| | | | | Nuclear |
|---|---|---|---|---|
| Age at the day | Gleason | Gleason | | Stat5 |
| of operation | grade | score | Stage | levels |
| 58 | (3 + 4) | 7 | T3a | 3 |
| 66 | (4 + 3) | 7 | T2b | 3 |
| 72 | (4 + 3) | 7 | T1c | 3 |
| 55 | (3 + 4) | 7 | T2b | 3 |
| 66 | (3 + 4) | 7 | T1c | 2 |
| 60 | (3 + 4) | 7 | T2a | 2 |
| 55 | (3 + 4) | 7 | T2a | 3 |
| 67 | (3 + 4) | 7 | T2a | 2 |

Samples of each prostate cancer were scored for nuclear Stat5 levels on a scale from 0 to 3 where 0 represented negative, 1 weak, 2 moderate and 3 strong immunostaining.

The specimens comprised de-identified excess tissue obtained after the completion of pathology diagnosis. A zero-sample prior to organ culture was formalin-fixed for each individual prostate cancer for the analysis of the nuclear Stat5a/b status. The prostate organ cultures were performed as described earlier (Ahonen et al., Endocrinology (2002), 143:228-238). Briefly, prostate cancer tissue was cut into approximately 1 mm³ pieces in a plain culture medium and transferred onto lens papers lying on stainless steel grids in petri dishes. The medium was phenol-free medium 199 with Earle's salts (Sigma) containing dialyzed 5% fetal calf serum, G-penicillin (100 IU/ml), streptomycin sulfate (100□l/ml), and glutamine (100 μg/ml). The basal medium also contained insulin (Novo Nordisk, Princeton, N.J.) (0.08 IU/ml), dexamethasone (Sigma) (100 nM) and DHT (100 nM), as previously established. The gas atmosphere was a mixture of O₂, CO₂, and N₂ (40:5:55), and the temperature was 37° C. Four parallel dishes each containing 20 prostate cancer explants were cultured per treatment group. The explants were cultured for 7 days, and the medium was changed every other day. Tissue explants were cultured in a medium containing the N6BAP or control compound C5 at indicated concentrations (C5: 100 m; N6BAP: 5, 10, 25, 50 and 100 μM) 7 days, after which the explants were fixed in formalin and analyzed for Stat5a/b activation and viability of prostate cancer epithelial compartment. Viability of the epithelial cells in the prostate cancer explants at the end of the cultures were scored by counting the viable epithelial cells per explant.

Nuclear Stat5a/b indexes were determined by counting the Stat5-positive cells per 500 epithelial cells in the cultured explants in each treatment group. Intensive positive immunostaining for nuclear Stat5 in explants cultured in the presence of the control compound C5 is observed, while N6BAP reduced the levels of nuclear Stat5 expression.

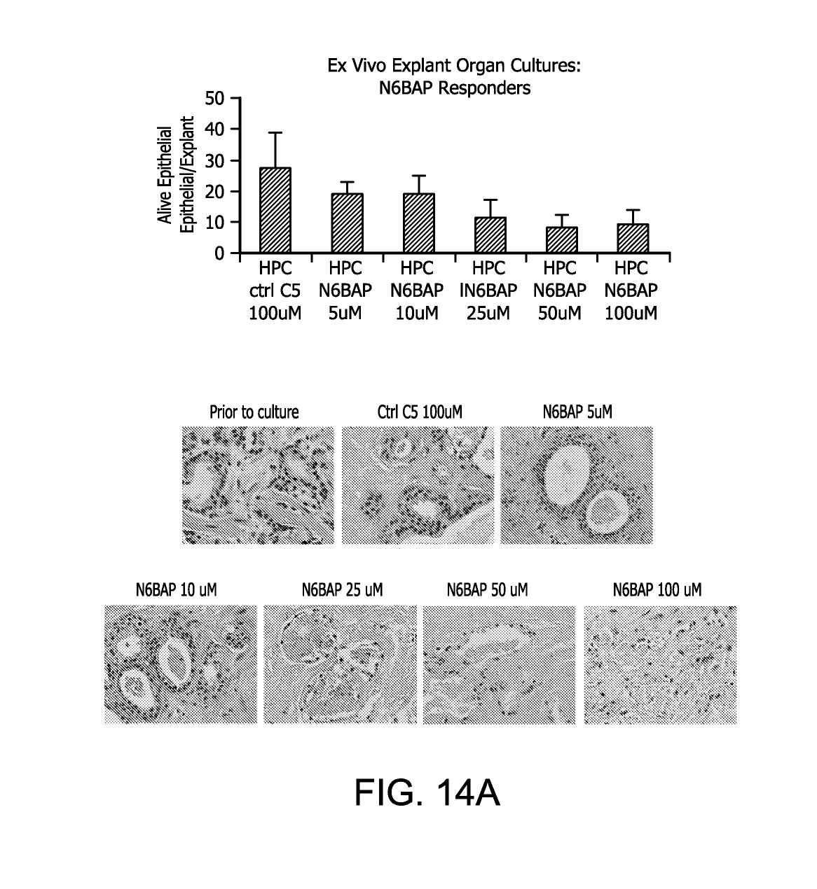

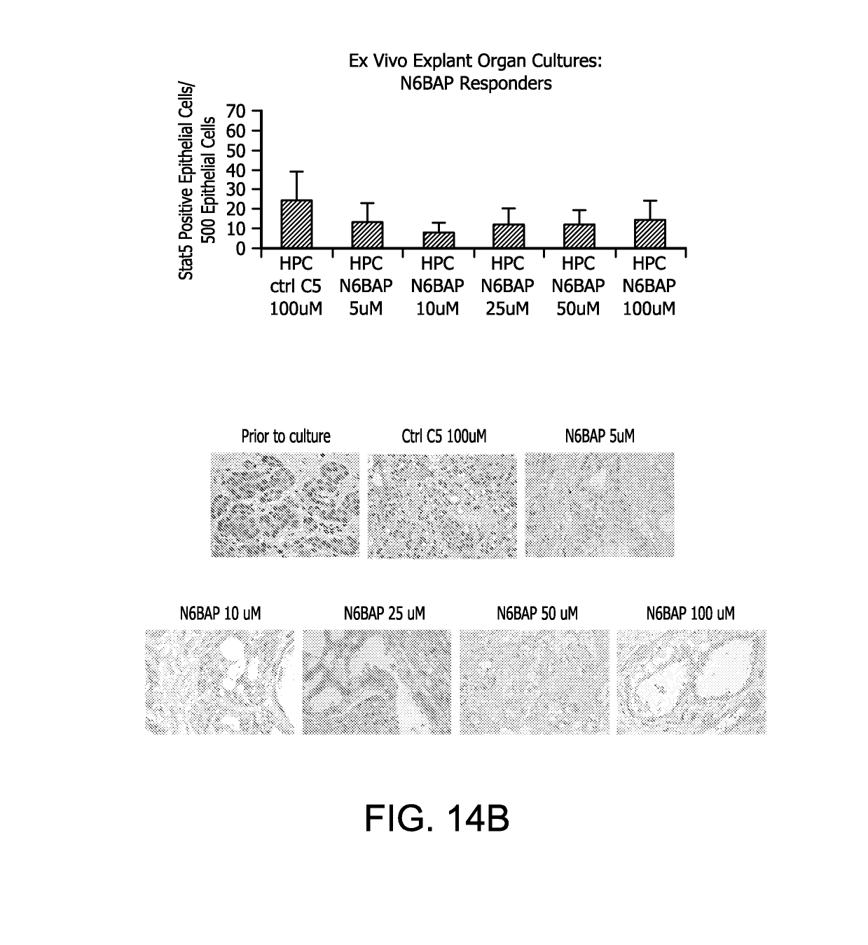

The results of the studies shown in the plots in FIGS. 14A (cell death) and 14B (nuclear active Stat5a/b level). The plots demonstrate that N6BAP induced extensive death of prostate cancer epithelium of clinical human prostate cancers in ex vivo explant organ cultures, and that N6BAP blocked nuclear translocation of Stat5 in these cultures. The cultures responded to N6BAP by excessive loss of viable acinar epithelium. Intensive positive immunostaining for nuclear Stat5 in explants cultured in the presence of the control compound C5 is observed, while N6BAP reduced the levels of nuclear Stat5 expression. The stained views shown in the panels beneath the plot in FIG. 14A comprise a representative histology of one individual. The stained views shown in the panels beneath the plot in FIG. 14A comprise a representative immunostaining of nuclear Stat5 in a human clinical prostate cancer that was responsive to N6BAP.

All references discussed herein are incorporated by reference. One skilled in the art will readily appreciate that the present invention is well adapted to carry out the objects and obtain the ends and advantages mentioned, as well as those inherent therein. The present invention may be embodied in other specific forms without departing from the spirit or essential attributes thereof and, accordingly, reference should be made to the appended claims, rather than to the foregoing specification, as indicating the scope of the invention.

1. A method of treating prostate cancer in a male in need of such treatment comprising administering to the male a therapeutically effective amount of N⁶-benzyladenosine-5′-monophosphate, or a pharmaceutically acceptable salt thereof.

2. The method according to claim 1, wherein the prostate cancer is organ-confined primary prostate cancer, locally invasive advanced prostate cancer, metastatic prostate cancer, castration-resistant prostate cancer, or recurrent castration-resistant prostate cancer.

3. (canceled)

4. A method of inhibiting prostate cancer cell growth comprising contacting prostate cancer cells with an amount of N⁶-benzyladenosine-5′-monophosphate, or a pharmaceutically acceptable salt thereof, effective to inhibit such cell growth.

5. The method according to claim 1 further comprising administering to said male at least one of radiation therapy and chemotherapy with an other chemotherapeutic agent effective against prostate cancer.

6. The method according to claim 5, wherein said other chemotherapeutic agent is selected from the group consisting of docetaxel, mitoxantrone, estramustine, doxorubicin, etoposide, vinblastine, paclitaxel, carboplatin, and vinorelbine, and combinations thereof.

7. The method according to claim 5, wherein said other chemotherapeutic agent is administered simultaneously with said N⁶-benzyladenosine-5′-monophosphate, or a pharmaceutically acceptable salt thereof.

8. The method according to claim 5, wherein said other chemotherapeutic agent is administered serially with said N⁶-benzyladenosine-5′-monophosphate, or a pharmaceutically acceptable salt thereof.

9. The method according to claim 7, wherein said other chemotherapeutic agent and said N⁶-benzyladenosine-5′-monophosphate, or a pharmaceutically acceptable salt thereof, are administered in the same dosage form.

10. A pharmaceutical composition for treatment of prostate cancer comprising N⁶-benzyladenosine-5′-monophosphate, or a pharmaceutically acceptable salt thereof, and at least one other chemotherapeutic agent effective against prostate cancer.

11. The composition according to claim 10, wherein said other chemotherapeutic agent is selected from the group consisting of docetaxel, mitoxantrone, estramustine, doxorubicin, etoposide, vinblastine, paclitaxel, carboplatin, and vinorelbine, and combinations thereof.

12. A The method according to claim 1, further comprising administering to said male an androgen ablation therapy.

13. The method according to claim 12, wherein the androgen ablation therapy comprises castration.

14. The method according to claim 12, wherein the androgen ablation therapy comprises administration of (i) at least one luteinizing hormone releasing hormone agonist, (ii) at least one anti-androgen, (iii) at least one inhibitor of prostate synthesis of androgenic steroids, or (iv) a combination of two or three of (i), (ii) and (iii).


SEQUENCE LISTING (1 sequence)

SEQ ID NO: 1 - DNA, 21 bp, Artificial Sequence
  Chemically synthesized probe
agatttctaggaattcaatcc